(19) United States
(12) Patent Application Publication
(10) Pub. No.: US 2009/0029375 A1
(43) Pub. Date: Jan. 29, 2009

(54) GENETIC MODELS FOR STRATIFICATION OF CANCER RISK

(75) Inventors: ELDON JUPE, NORMAN, OK (US); CRAIG SHIMASAKI, EDMOND, OK (US); DAVID RALPH, EDMOND, OK (US)

Correspondence Address: FULBRIGHT & JAWORSKI L.L.P., 600 CONGRESS AVE., SUITE 2400, AUSTIN, TX, 78701, US

(73) Assignee: Intergenetics, Inc.

(21) Appl. No.: 12/171,819

(22) Filed: Jul. 11, 2008

Related U.S. Application Data
(60) Provisional application No. 60/949,172, filed on Jul. 11, 2007.
(60) Provisional application No. 60/951,110, filed on Jul. 20, 2007.

(51) Int. Cl. C12Q 1/68; C40B 40/08

(52) U.S. Cl. 435/6; 506/17

(57) ABSTRACT

The present invention provides new methods for the assessment of cancer risk in the general population. These methods utilize particular alleles of in multiple selected genes to identify individuals with increased or decreased risk of breast cancer. In addition, personal history measures such as age and family history are used to further refine the analysis. Using such methods, it is possible to reallocate healthcare costs in cancer screening to patient subpopulations at increased cancer risk. It also permits identification of candidates for cancer prophylactic treatment.

The present application claims benefit of priority to U.S. Provisional Application Ser. No. 60/949,172, filed Jul. 11, 2007 and U.S. Provisional Application Ser. No. 60/951,110, filed Jul. 20, 2007, the entire contents of both which are hereby incorporated by reference.

The government owns rights in the present invention pursuant to grant number DAMD17-01-1-0358 from the United States Army Breast Cancer Research Program, and grant numbers AR992-007, AR01.1-050 and AR05.1025 from the Oklahoma Center for the Advancement of Science and Technology (OCAST).

### BACKGROUND OF THE INVENTION

1. Field of the Invention

The present invention relates generally to the fields of oncology and genetics. More particularly, it concerns use of multivariate analysis of genetic alleles constituting genotypes to determine genotypes and combinations of genotypes associated with low, intermediate and high risk of particular cancers. These risk alleles are used to screen patient samples, evaluation of incremental and lifetime risk of developing cancer, and efficiently direct patients towards prediagnostic cancer risk management and prophylaxis.

2. Description of Related Art

For patients with cancer, early diagnosis and treatment are the keys to better outcomes. In 2001, there are expected to be 1.25 million persons diagnosed with cancer in the United States. Tragically, in 2001 over 550,000 people are expected to die of cancer. To a very large extent, the difference between life and death for a cancer patient is determined by the stage of the cancer when the disease is first detected and treated. For those patients whose tumors are detected when they are relatively small and confined, the outcomes are usually very good. Conversely, if a patient's cancer has spread from its organ of origin to distant sites throughout the body, the patient's prognosis is very poor regardless of treatment. The problem is that tumors that are small and confined usually do not cause symptoms. Therefore, to detect these early stage cancers, it is necessary to continually screen or examine people without symptoms of illness. In such apparently healthy people, cancers are actually quite rare. Therefore it is necessary to screen a large number of people to detect a small number of cancers. As a result, annual or regularly administered cancer-screening tests are relatively expensive to administer in terms of the number of cancers detected per unit of healthcare expenditure.

A related problem in cancer screening is derived from the reality that no screening test is completely accurate. All tests deliver, at some rate, results that are either falsely positive (indicate that there is cancer when there is no cancer present) or falsely negative (indicate that no cancer is present when there really is a tumor present). Falsely positive cancer screening test results create needless healthcare costs because such results demand that patients receive follow-up examinations, frequently including biopsies, to confirm that a cancer is actually present. For each falsely positive result, the costs of such follow-up examinations are typically many times the costs of the original cancer-screening test. In addition, there are intangible or indirect costs associated with falsely positive screening test results derived from patient discomfort, anxiety and lost productivity. Falsely negative results also have associated costs. Obviously, a falsely negative result puts a patient at higher risk of dying of cancer by delaying treatment. To counter this effect, it might be reasonable to increase the rate at which patients are repeatedly screened for cancer. This, however, would add direct costs of screening and indirect costs from additional falsely positive results. In reality, the decision on whether or not to offer a cancer screening test hinges on a cost-benefit analysis in which the benefits of early detection and treatment are weighed against the costs of administering the screening tests to a largely disease free population and the associated costs of falsely positive results. In addition, many advanced screening and imaging methods exist that are more accurate than general screening tests, but the costs for administering these tests using these advanced imaging tools is many times more expensive.

Another related problem concerns the use of chemopreventative drugs for cancer. Basically, chemopreventatives are drugs that are administered to prevent a patient from developing cancer. While some chemopreventative drugs may be effective, such drugs are not appropriate for all persons because the drugs have associated costs and possible adverse side effects (Reddy and Chow, 2000). Some of these adverse side effects may be life threatening. Therefore, decisions on whether to administer chemopreventative drugs are also based on a risk-benefit analysis. The central question is whether the benefits of reduced cancer risk outweigh the associated drug risks and costs of the chemopreventative treatment.

The risk-benefit balance has to be favorable for prescribing a preventative drug and it is not favorable for an individual who is not at increased risk for developing cancer, where it is for an individual who is at increased risk. One problem is being able to effectively identify individuals that are at higher-than-average risk for developing cancer.

Currently, an individual's age is the most important factor in determining if a particular cancer-screening test should be offered to a patient. Truly, cancer is a rare disease in the young and a fairly common ailment in the elderly. The problem arises in screening and preventing cancers in the middle years of life when cancer can have its greatest negative impact on life expectancy and productivity. In the middle years of life, cancer is still fairly uncommon. Therefore, the costs of cancer screening and prevention can still be very high relative to the number of cancers that are detected or prevented. Decisions on when to begin screening also may be influenced by personal history or family history measures. Unfortunately, appropriate informatic tools to support such decision-making are not yet available for most cancers.

A common strategy to increase the effectiveness and economic efficiency of cancer screening and chemoprevention in the middle years of life is to stratify individuals' cancer risk and focus the delivery of screening and prevention resources on the high-risk segments of the population. Two such tools to stratify risk for breast cancer are termed the Gail Model and the Claus Model (Costantino et al., 1999; McTiernan et al., 2001). The Gail model is used as the “Breast Cancer Risk-Assessment. Tool” software provided by the National Cancer Institute of the National Institutes of Health on their web site. Neither of these breast cancer models utilizes genetic markers as part of their inputs. Furthermore, while both models are steps in the right direction, neither the Gail nor Clause models have the desired predictive power or discriminatory accuracy to truly optimize the delivery of breast cancer screening or chemopreventative therapies.

These issues and problems could be reduced in scope or even eliminated if it were possible to stratify or differentiate a given individual's risk from cancer more accurately than is now possible. If a precise measure of actual risk could be accurately determined, it would be possible to concentrate cancer screening and chemopreventative efforts in that segment of the population that is at highest risk. With accurate stratification of risk and concentration of effort in the high-risk population, fewer screening tests or more advanced screening tests that may be more expensive would be directed toward the higher risk segment of individuals to detect a greater number of cancers at an earlier and more treatable stage. Fewer screening tests would mean lower test administrative costs and fewer falsely positive results. A greater number of cancers detected would mean a greater net benefit to patients and other concerned parties such as health care providers. Similarly, chemopreventative drugs would have a greater positive impact by focusing the administration of these drugs to a population that receives the greatest net benefit.

### SUMMARY OF THE INVENTION

Thus, in accordance with the present invention, there is provided a method for assessing a female subject's risk for developing breast cancer comprising determining, in a sample from the subject, the allelic profile of more than one SNP selected from the group consisting of ACACA (IVS17) T→C, ACACA (5′UTR) T→C, ACACA (PIII) TAG, COMT (rs4680) A→G, CYP19 (rs10046) T→C, CYP1A1 (rs4646903) T→C, CYP1B1 (rs 1800440) A→G, EPHX (rs1051740) T→C, TNFSF6 (rs763110) C→T, IGF2 (rs2000993) G→A, INS (rs3842752) C→T, KLK10 (rs3745535) G→T, MSH6 (rs3136229) G→A, RAD51L3 (rs4796033) G→A, XPC (rs2228000) C→T, and XRCC2 (rs3218536) G→A, including 3, 4, 5, 6, 7, 8, 9, 10, 11, 12, 13, 14, 15, 16, 17, 18, 19, 20, 21 or 22 SNPs in 19 genes. The method may further comprise determining the allelic profile of at least one additional SNP selected from the group consisting of CYP11B2 (rs1799998) T→C, CYP1B1 (rs10012) C→G, ESR1 (rs2077647) T→C, SOD2 (aka MnSOD, rs1799725) T→C, VDR (rs7975232) T→G, and ERCC5 (rs17655) G→C.

The method may also further comprise assessing one or more aspects of the subject's personal history, such as age, ethnicity, reproductive history, menstruation history, use of oral contraceptives, body mass index, alcohol consumption history, smoking history, exercise history, diet, family history of breast cancer or other cancer including the age of the relative at the time of their cancer diagnosis, and a personal history of breast cancer, breast biopsy or DCIS, LCIS, or atypical hyperplasia. Age may comprise stratification into a young age group of age 30-44 years, middle age group of age 45-54 years, and an old age group of 55 years and older. Alternatively, age may comprising stratification in 30-49 years and 50-69 years, or 50 and older.

The step of determining the allelic profile may be achieved by amplification of nucleic acid from the sample, such as by PCR, including chip-based assays using primers and primer pairs specific for alleles of the genes. The method may also further comprising cleaving the amplified nucleic acid. Samples may be derived from oral tissue collected by lavage or blood. The method may also further comprise making a decision on the timing and/or frequency of cancer diagnostic testing for the subject; and/or making a decision on the timing and/or frequency of prophylactic cancer treatment for the subject.

In another embodiment, there is provided a nucleic acid microarray comprising nucleic acid sequences corresponding to genes at least one of the alleles for each of ACACA (IVS17) T→C, ACACA (5′UTR) T→C, ACACA (PIII) T→G, COMT (rs4680) A→G, CYP19 (rs10046) T→C, CYP1A1 (rs4646903) T→C, CYP1B1 (rs1800440) A→G, EPHX (rs1051740) T→C, TNFSF6 (rs763110) C→T, IGF2 (rs2000993) G→A, INS (rs3842752) C→T, KLK10 (rs3745535) G→T, MSH6 (rs3136229) G→A, RAD51L3 (rs4796033) G→A, XPC (rs2228000) C→T, and XRCC2 (rs3218536) G→A. The nucleic acid sequences may comprise sequences for both alleles for each of the genes.

In still yet another embodiment, there is provided a method for determining the need for routine diagnostic testing of a female subject for breast cancer comprising determining, in a sample from the subject, the allelic profile of more than one SNP selected from the group consisting of ACACA (IVS17) T→C, ACACA (5′UTR) T→C, ACACA (PIII) T+G, COMT (rs4680) A→G, CYP19 (rs10046) T→C, CYP1A1 (rs4646903) T→C, CYP1B1 (rs1800440) A→G, EPHX (rs1051740) T→C, TNFSF6 (rs763110) C→T, IGF2 (rs2000993) G→A, INS (rs3842752) C→T, KLK10 (rs3745535) G→T, MSH6 (rs3136229) G→A, RAD51L3 (rs4796033) G→A, XPC (rs2228000) C→T, and XRCC2 (rs3218536) G→A.

In yet a further embodiment, there is provided a method for determining the need of a female subject for prophylactic anti-breast cancer therapy comprising determining, in a sample from the subject, the allelic profile of more than one SNP selected from the group consisting of ACACA (IVS17) T→C, ACACA (5′UTR) T→C, ACACA (PIII) T→G, COMT (rs4680) A→G, CYP19 (rs10046) T→C, CYP1A1 (rs4646903) T→C, CYP1B1 (rs1800440) A→G, EPHX (rs1051740) T→C, TNFSF6 (rs763110) C→T, IGF2 (rs2000993) G→A, INS (rs3842752) C→T, KLK10 (rs3745535) G→T, MSH6 (rs3136229) G→A, RAD51L3 (rs4796033) G→A, XPC (rs2228000) C→T, and XRCC2 (rs3218536) G→A.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows an overview of the components comprising the algorithm of the integrated predictive model. The flow of analyses performed on the genotyping information is dependent on the patient's current age and history of a first degree relative with breast cancer.

FIGS. 2A-C show an illustration of the OncoVue® Multifactorial Risk Estimator.

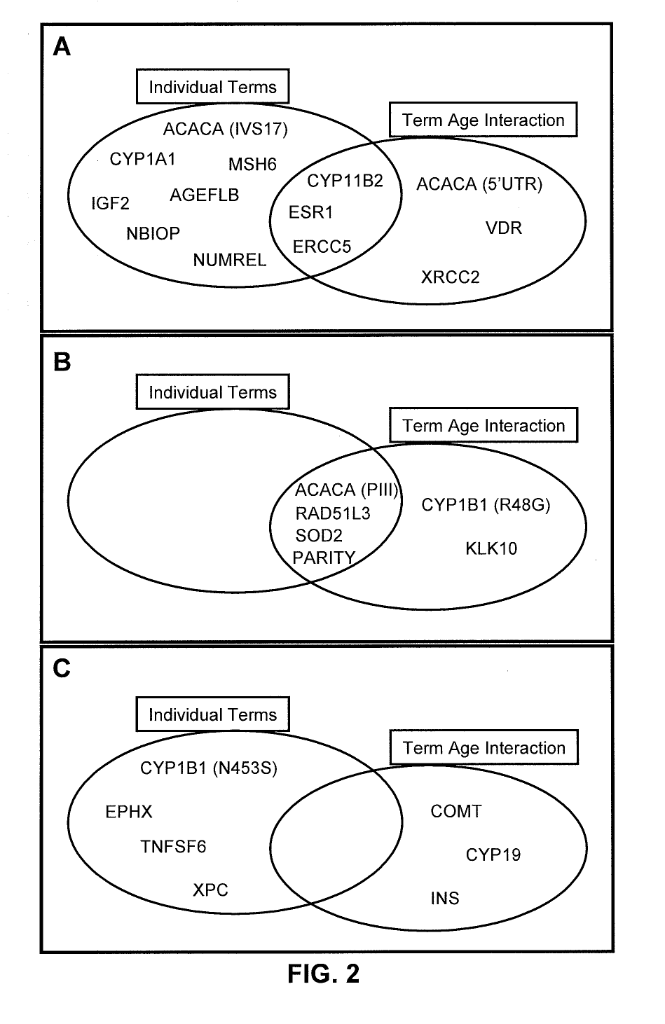

In each panel, the left ellipse shows the individual terms in the model and the right ellipse shows the terms interacting with age. The overlapping region in the middle shows terms included both individually and interacting with age. FIG. 2A is for all ages, FIG. 2B is for ages 30-49 without a first degree relative and FIG. 2C is for ages 30-49 with a first degree relative.

### DESCRIPTION OF ILLUSTRATIVE EMBODIMENTS

Despite considerable progress in cancer therapy, cancer mortality rates continue to be high. Generally, the poor prognosis of many cancer patients derives from the failure to identify the disease at an early stage, i.e., before metastasis has occurred. While not trivial, treatment of organ confined primary tumors is far more likely to be successful than any treatment for advanced, disseminated malignancies.

In order to affect early diagnosis of cancer, at a time when patients still appear healthy, it is necessary to screen large numbers of individuals. However, the costs associated with such testing, and the unnecessary follow-ups occasioned by false positive results, are prohibitive. Thus, it is necessary to find better ways of assessing cancer risk in the general population and concentrating preventative and early detection efforts on those individuals at highest risk.

### I. THE PRESENT INVENTION

In accordance with the present invention, the inventors have identified alleles for Single Nucleotide Polymorphisms (SNPs) and other genetic variations that are associated with varying levels of risk for a diagnosis of breast cancer. A SNP is the smallest unit of genetic variation. It represents a position in a genome where individuals of the same species may have alternative nucleotides present at the same site in their DNA sequences. It could be said that our genes make us human, but our SNPs make us unique individuals. An allele is a particular variant of a gene. For example, some individuals may have the DNA sequence, AAGTCCG, in some arbitrary gene. Other individuals may have the sequence, AAGTTCG, at the same position in the same gene. Notice that these DNA sequences are the same except at the underlined position where some people have a “C” nucleotide while others have a “T” nucleotide. This is the site of a SNP. It is said that some people carry the C allele of this SNP, while others carry the T allele.

Except for those genes on the sex chromosomes and in the mitochondrial genome, there are two copies of every gene in every cell in the body. A child inherits one copy of each gene from each parent. A person could have two C alleles of the fictitious SNP described above. This person would carry the genotype C/C at this SNP. Alternatively, a person could have the genotype T/T at this SNP. As in both of these examples, if someone carries two identical copies of a portion of a potentially variant portion of a gene, they are referred to as homozygous for this gene or portion of a gene. Obviously, some people will carry two different alleles of this gene having the genotype, C/T or T/C, and will be termed heterozygous for this SNP. Lastly, some genetic variation may involve more than one nucleotide position. Common examples of such variation, and ones that are relevant to this invention, are polymorphisms where there have been insertions or deletions of one or more nucleotides in one allele of a gene relative to the alternative allele(s).

In addition to genetic variation, the inventors have examined the interaction between age and genetic variation to better estimate risk of breast cancer. They have also begun to examine ethnic affiliation and family history of cancer as additional variables to better estimate breast cancer risk. Age, gender, ethnic affiliation and family medical history are all examples of personal history measures. Other examples of personal history measures include reproductive history, menstruation history, use of oral contraceptives, body mass index, smoking and alcohol consumption history, and exercise and diet.

In the experiments disclosed herein, the inventors report the examination of alleles of numerous genetic polymorphisms. Polymorphisms were assayed by standard techniques to detect these SNPs including Allele Specific Primer Extension (ASPE), Restriction Fragment Length Polymorphisms (RFLPs) or simple length polymorphisms in gene specific PCR products. All of the polymorphisms examined have been described previously in the peer reviewed scientific literature as having some functional activity or association with disease, usually cancer. The OncoVue® test described here examines 22 SNPs in 19 genes located on 13 different-chromosomes (1, 2, 3, 6, 7, 8, 11, 12, 13, 15, 17, 19 and 22). The 19 genes are involved in the following 7 major cellular pathways with the number of genes in each pathway shown below:

Steroid hormone metabolism (6)

DNA Repair (6)

Growth Factors (3)

Cell Cycle/Apoptosis (1)

Extracellular matrix (1)

Free Radical Scavenger (1)

Xenobiotic metabolism (1)*

*—refers to detoxification of pollutants, drugs, etc., that are foreign to the organism.

The inventors' hypothesis was that by examining these polymorphisms in very large associative studies one would find certain genotypes and combinations of genotypes that were much more informative for predicting cancer risk than could have been predicted a priori. In fact, it now has been determined that certain genotypes and combinations of genotypes are associated with extraordinary risk of breast cancer. So high is the genetically inherited risk of breast cancer in individuals carrying certain genotypes and combinations of genotypes, that their risk distorts the apparent breast cancer risk in the population at large. Thus, surprisingly, the large majority of women are actually at much less than “average” risk from breast cancer. Such dramatic findings were unexpected even by the inventors when these experiments were designed. These results provide a means of reallocating breast cancer screening and chemoprevention resources to concentrate on a relatively small portion of the total population at highest risk of breast cancer, thus facilitating better patient outcomes at lower overall healthcare costs.

### II. TARGET GENES AND ALLELES

Table 1, below, provides a listing of the genes, the specific genetic polymorphisms examined in the present study, and a literature citation. The letters in parentheses are abbreviations for these polymorphisms that will be used throughout the remainder of this text.

Some of these polymorphisms have been discussed in the literature in depth in perhaps dozens of scientific publications. While the scientific literature suggests that many of these polymorphisms may be associated with very modest changes in cancer risk, or are associated with larger variations in risk within a small subset of the population, many of these polymorphisms are controversial in the scientific literature, with some studies finding no associated change in relative cancer risk. Formally, in genetic terms, these common SNP genotypes individually have low penetrance for the breast cancer phenotype, but when occurring together create complex genotypes with very high penetrance for the breast cancer phenotype. The inventors note that their hypothesis for cancer predisposition is consistent with that of a complex multi-gene phenomenon, as has been discussed by others (Lander and Schork, 1994), and is in agreement with the long-standing observation that cancers in general, and breast cancer in particular, are complex diseases. However, these particular gene combinations have not previously been identified as being associated with risk of breast or any other cancer. The model developed integrates information from multiple genes and personal history measures to evaluate risk of developing breast cancer. The genetic effects that are incorporated into the model were identified in multivariate logistic regression analyses as significantly associated with breast cancer risk. In a given age group, the collective consideration of 10-16 markers has predictive value that exceeds any single term in other words the whole is greater than any single part. Beyond this non-parametric analyses of candidate genes have identified oligogenic combinations associated with breast cancer risk (WO2003/025141; WO2005/024067). An initial published study examined polymorphisms in ten genes and identified a total of 69 two- and three-gene combinations significantly associated with breast cancer risk (Aston et al., 2005). This represented over thirty times as many significant associations as would be expected by random chance. The odds ratios (ORs) of these oligogenic combinations ranged from 0.5 to 5.9. Thus, consideration of multiple genes in risk prediction for complex disease can far exceed the predictive value of any given single gene.

### III. SAMPLE COLLECTION AND PROCESSING

A. Sampling

In order to assess the genetic make-up of an individual, it is necessary to obtain a nucleic acid-containing sample. Suitable tissues include almost any nucleic acid containing tissue, but those most convenient include oral tissue or blood. For those DNA specimens isolated from peripheral blood specimens, blood was collected in heparinized syringes or other appropriate vessel following venipuncture with a hypodermic needle. Oral tissue may advantageously be obtained from a mouth rinse. Oral tissue or buccal cells may be collected with oral rinses, e.g., with “Original Mint” flavor Scope™ mouthwash. Typically, a volunteer participant would vigorously swish 10-15 ml of mouthwash in their mouth for 10-15 seconds. The volunteer would then spit the mouthwash into a 50 ml conical centrifuge tube (for example Fisherbrand disposable centrifuge tubes with plug seal caps (catalog #05-539-6)) or other appropriate container.

B. Processing of Nucleic Acids

Genomic DNA was isolated and purified from the samples collected as described below using the PUREGENE™ DNA isolation kit manufactured by Gentra Systems of Minneapolis, Minn.

A number of different materials are used in accordance with the present invention. These include primary solutions used in DNA Extraction (Cell Lysis Solution, Gentra Systems Puregene, and Cat. # D-50K2, 1 Liter; Protein Precipitation Solution, Gentra Systems Puregene, Cat. # D-50K3, 350 ml; DNA Hydration Solution, Gentra Systems Puregene, Cat. # D-500H, 100 ml) and secondary solutions used in DNA Extraction (Proteinase K enzyme, Fisher Biotech, Cat. # BP1700, 100 mg powder; RNase A enzyme, Amresco, Cat. # 0675, 500 mg powder; Glycogen, Fisher Biotech, Cat. # BP676, 5 gm powder, 2-propanol (isopropanol), Fisher Scientific, Cat. # A451, 1 Liter; TE Buffer Solution pH 8.0, Amresco, Cat. # E112, 100 ml; 95% Ethyl Alcohol, AAPER Alcohol & Chemical Co., 5 Liters).

The exemplified DNA extraction procedure involves five basic steps, as discussed below:
Preliminary Procedures: Buccal samples should be processed within 7 days of collection. The DNA is stable in mouthwash at room temperature, but may degrade if left longer than a week before processing.
Cell Lysis and RNase A Treatment: Samples are centrifuged (50 ml centrifuge tube containing the buccal cell sample) at 3000 rpm (or 2000×g) for 10 minutes using a large capacity (holds 20-50 ml or 40-15 ml centrifuge tubes) refrigerated centrifuge. Immediately pour off the supernatant into a waste bottle, leaving behind roughly 100 μl of residual liquid and the buccal cell pellet at the bottom of the 50 ml tube. Be aware that loose pellets will result if samples are left too long after centrifugation before discarding the liquid. Vortex (using a Vortex Genie at high speed) for 5 seconds to resuspend the cells in the residual supernatant. This greatly facilitates cell lysis (below). Pipette (use a pipette aide and a 10 ml pipette) 1.5 ml of Cell Lysis Solution into the 50 ml tube to resuspend the cells, and then vortex for 5 seconds to maximize contact between cells and cell lysis solution. If necessary, new samples may need to be stored longer than a week before finishing the whole DNA extraction process. If so, one needs to process the samples to the point of adding Cell Lysis Solution and store the samples at 4° C. The samples will easily be kept viable for months. Do not store unprocessed samples at 4° C., as this has been shown to prevent the preparation of DNA that produces an easily executed PCR. Using a 20 μl Pipetman and 250 μl pipettes, add 15 μl of Proteinase K (10 mg/ml) enzyme into each sample tube, releasing Proteinase K directly into the cell lysate solution of each tube. No part of the Pipetman should touch sample tube—only the pipette tips. Change pipette tip with each sample tube. Vortex briefly to mix. Incubate the cell lysate in the 50 ml tube at 55° C. for 1 hour. The enzyme will not activate until around 55° C., so make sure incubator is near that temperature before starting. It is permissible to incubate longer if needed, even overnight. Pipette 5 μl of RNase A (5 mg/ml) enzyme directly into the cell lysate solution of each 50 ml sample tube. This is required because of the relatively small volume of the enzyme. Change pipette tips for every new sample. Mix the sample by inverting the tube gently 25 times, and then incubate in the water bath at 37° C. for 15 minutes.
Protein Precipitation The sample should be cooled to room temperature. At this point, sample may sit for an hour if needed. Using the pipette aide and 5 ml pipettes, add 0.5 ml of Protein Precipitation Solution to each 50 ml sample tube of cell lysate. Vortex samples for 20 seconds to mix the Protein Precipitation Solution uniformly with the cell lysate. Place 50 ml sample tube in an ice bath for a minimum of 15 minutes, preferably longer. This ensures that the cell protein will form a tight pellet when you centrifuge (next step). Centrifuge at 3000 rpm (2000×g) for 10 minutes, having the centrifuge refrigerated to 4° C. The precipitated proteins should form a tight, white or green pellet (it may appear green if mint mouthwash was used to collect the buccal samples).
DNA Precipitation: While waiting for the centrifuge to finish, prepare enough sterile 15 ml centrifuge tubes to accommodate your samples. Add 5 μl of glycogen (10 mg/ml) to each tube, forming a bead of liquid near the top. Then add 1.5 ml of 100% 2-propanol to each tube. Carefully pour the supernatant containing the DNA into the prepared 15 ml tubes, leaving behind the precipitated protein pellet in the 50 ml tube. If the pellet is loose you may have to pipette the supernatant out, getting as much clear liquid as possible. Pellet may be loose because the sample was not chilled long enough or may need to be centrifuged longer. Nothing but clear greenish liquid should go into the new 15 ml tube. Be careful that the protein pellet does not break loose as you pour. Record on new tube the correct sample number as was on the 50 ml tube. Discard the 50 ml tube. Mix the 15 ml sample tube by inverting gently 50 times. Rough handling may shear DNA strands. Clean white strands clumping together should be observed. Keep at room temperature for at least 5 minutes. Centrifuge at 3000 rpm (2000×g) for 10 minutes. The DNA may or may not be visible as a small white pellet, depending on yield. If the pellet is any other color, the sample has contamination. If there is apparent high yield, it may also point to contamination. Pour off the supernatant into a waste bottle, being careful not to let the DNA dislodge and slide out with the liquid. Invert the open 15 ml sample tubes over a clean absorbent paper towel to drain out remaining liquid. Let sit for 5 minutes. Invert tubes right side back up, put caps back on and set them in holding tray (Styrofoam tray the 15 ml tubes were shipped in) with numbered side facing away. Add 1.5 ml of 70% ethanol to each tube. Invert the tubes several times to wash the DNA pellet. Centrifuge at 3000 rpm (2000×g) for 3 minutes. Carefully pour off the ethanol. Invert the sample tube onto a paper towel and let air dry no longer than 15 minutes before resuspending the DNA using a hydration solution. If the DNA is allowed to dry out completely, it will increase the difficulty of rehydrating it.
DNA Hydration: Depending on the size of the resulting DNA pellet, add between 50-200 μl of DNA Hydration Solution to the 15 ml sample tube. If the tube appears to have no DNA, use 50 μl. If it appears to have some, but not a lot, use 100 μl. With a good-sized pellet, 150-200 μl can be used. This is important because the concentration of DNA affects the results of the PCR experiment, and one does not want to dilute the DNA too much. The optimal concentration of DNA is around 100 ng/μl. Allow the DNA to hydrate by incubating at room temperature overnight or at 65° C. for 1 hour. Tap the tube periodically or place on a rotator to aid in dispersing DNA (this helps if the DNA was allowed to dry out completely, but normally it is not required). For storage, sample should be centrifuged briefly and transferred to a cross-linked or UV radiated 1.5 ml centrifuge tube (that was previously autoclaved). Store genomic DNA sample at 4° C. For long-term storage, store at −20° C.
   While suitable substitute procedures may suffice, following the preceding protocol will ensure the fidelity of the results.

C. cDNA Production

In one aspect of the invention, it may be useful to prepare a cDNA population for subsequent analysis. In typical cDNA production, mRNA molecules with poly(A) tails are potential templates and will each produce, when treated with a reverse transcriptase, a cDNA in the form of a single-stranded molecule bound to the mRNA (cDNA:mRNA hybrid). The cDNA is then converted into double-stranded DNA by DNA polymerases such as DNA Pol I (Klenow fragment). Klenow polymerase is used to avoid degradation of the newly synthesized cDNAs. To produce the template for the polymerase, the mRNA must be removed from the cDNA:mRNA hybrid. This is achieved either by boiling or by alkaline treatment (see lecture notes on the properties of nucleic acids). The resulting single-stranded cDNA is used as the template to produce the second DNA strand. As with other polymerases, a double-stranded primer sequence is needed and this is fortuitously provided during the reverse transcriptase synthesis, which produces a short complementary tail at the 5′ end of the cDNA. This tail loops back onto the ss cDNA template (the so-called “hairpin loop”) and provides the primer for the polymerase to start the synthesis of the new DNA strand producing a double stranded cDNA (ds cDNA). A consequence of this method of cDNA synthesis is that the two complementary cDNA strands are covalently joined through the hairpin loop. The hairpin loop is removed by use of a single strand specific nuclease (e.g., S1 nuclease from Aspergillus oryzae).

Kits for cDNA synthesis (SMART RACE cDNA Amplification Kit; Clontech, Palo Alto, Calif.). It also is possible to couple cDNA with PCR™, into what is referred to as RT-PCR™. PCR™ is discussed in greater detail below.

### IV. DETECTION METHODS

Once the sample has been properly processed, detection of sequence variation is required. Perhaps the most direct method is to actually determine the sequence of either genomic DNA or cDNA and compare these to the known alleles. This can be a fairly expensive and time-consuming process. Nevertheless, this is the lead technology of numerous bioinformatics companies with interests in SNPs including such firms as Perlegen, Genizon Biosciences, Celera, and Genaissance, and the technology is available to do fairly high volume sequencing of samples. A variation on the direct sequence determination method is the Gene Chip™ method as advanced by Affymetrix. Such chips are discussed in greater detail below. Competing with Affymetrix, Illumina has recently developed a number of high throughput SNP genotyping technologies.

Older technologies that continue to have some commercially viable applications include the TAQman Assay developed by Perkin Elmer, the SNP-IT™ (SNP-Identification Technology) developed by Orchid BioSciences, the MassARRAY™ system developed by Sequenom, the READIT™ SNP/Genotyping System (U.S. Pat. No. 6,159,693) developed by Promega and the Invader OS™ system developed by Third Wave Technologies. Finally, there are a number of forensic DNA testing labs and many research labs that still use gene-specific PCR, followed by restriction endonuclease digestion and gel electrophoresis (or other size separation technology) to detect RFLPs. The point is that, how one detects sequence variation (SNPs) is not important in the estimation of cancer risk. The key is the genes and polymorphisms that one examines.

As an alternative SNP detection technology to RFLP, genotypes were determined by Allele Specific Primer Extension (ASPE) coupled to a microsphere-based technical readout. Many accounts of SNP genotyping using microsphere-based methods have been published in the scientific literature. The method is being used as an alternative to RFLP and closely resembles that of Ye et al. (2001). This technology was implemented through the Luminex™-100 microsphere detection platform (Luminex, Austin, Tex.) using oligonucleotide labeled microspheres purchased from MiraiBio, Inc. (Alameda, Calif.).

The following materials and methodologies relate to the present invention, and are therefore described in some detail.

A. Chips

As discussed above, one convenient approach to detecting variation involves the use of nucleic acid arrays placed on chips. This technology has been widely exploited by companies such as Affymetrix, and a large number of patented technologies are available. Specifically contemplated are chip-based DNA technologies such as those described by Hacia et al. (1996) and Shoemaker et al. (1996). These techniques involve quantitative methods for analyzing large numbers of sequences rapidly and accurately. The technology capitalizes on the complementary binding properties of single stranded DNA to screen DNA samples by hybridization (Pease et al., 1994; Fodor et al., 1991).

Basically, a DNA array or gene chip consists of a solid substrate to which an array of single-stranded DNA molecules has been attached. For screening, the chip or array is contacted with a single-stranded DNA sample, which is allowed to hybridize under stringent conditions. The chip or array is then scanned to determine which probes have hybridized. In a particular embodiment of the instant invention, a gene chip or DNA array would comprise probes specific for chromosomal changes evidencing the predisposition towards the development of a neoplastic or preneoplastic phenotype. In the context of this embodiment, such probes could include PCR products amplified from patient DNA synthesized oligonucleotides, cDNA, genomic DNA, yeast artificial chromosomes (YACs), bacterial artificial chromosomes (BACs), chromosomal markers or other constructs a person of ordinary skill would recognize as adequate to demonstrate a genetic change.

A variety of gene chip or DNA array formats are described in the art, for example U.S. Pat. Nos. 5,861,242 and 5,578,832, which are expressly incorporated herein by reference. A means for applying the disclosed methods to the construction of such a chip or array would be clear to one of ordinary skill in the art. In brief, the basic structure of a gene chip or array comprises: (1) an excitation source; (2) an array of probes; (3) a sampling element; (4) a detector; and (5) a signal amplification/treatment system. A chip may also include a support for immobilizing the probe.

In particular embodiments, a target nucleic acid may be tagged or labeled with a substance that emits a detectable signal, for example, luminescence. The target nucleic acid may be immobilized onto the integrated microchip that also supports a phototransducer and related detection circuitry. Alternatively, a gene probe may be immobilized onto a membrane or filter, which is then attached to the microchip or to the detector surface itself. In a further embodiment, the immobilized probe may be tagged or labeled with a substance that emits a detectable or altered signal when combined with the target nucleic acid. The tagged or labeled species may be fluorescent, phosphorescent, or otherwise luminescent, or it may emit Raman energy or it may absorb energy. When the probes selectively bind to a targeted species, a signal is generated that is detected by the chip. The signal may then be processed in several ways, depending on the nature of the signal.

The DNA probes may be directly or indirectly immobilized onto a transducer detection surface to ensure optimal contact and maximum detection. The ability to directly synthesize on or attach polynucleotide probes to solid substrates is well known in the art. See U.S. Pat. Nos. 5,837,832 and 5,837,860, both of which are expressly incorporated by reference. A variety of methods have been utilized to either permanently or removably attach the probes to the substrate. Exemplary methods include: the immobilization of biotinylated nucleic acid molecules to avidin/streptavidin coated supports (Holmstrom, 1993), the direct covalent attachment of short, 5′-phosphorylated primers to chemically modified polystyrene plates (Rasmussen et al., 1991), or the precoating of the polystyrene or glass solid phases with poly-L-Lys or poly L-Lys, Phe, followed by the covalent attachment of either amino- or sulfhydryl-modified oligonucleotides using bi-functional crosslinking reagents (Running et al., 1990; Newton et al., 1993). When immobilized onto a substrate, the probes are stabilized and therefore may be used repeatedly. In general terms, hybridization is performed on an immobilized nucleic acid target or a probe molecule is attached to a solid surface such as nitrocellulose, nylon membrane or glass. Numerous other matrix materials may be used, including reinforced nitrocellulose membrane, activated quartz, activated glass, polyvinylidene difluoride (PVDF) membrane, polystyrene substrates, polyacrylamide-based substrate, other polymers such as poly(vinyl chloride), poly(methyl methacrylate), poly(dimethyl siloxane), and photopolymers (which contain photoreactive species such as nitrenes, carbenes and ketyl radicals) capable of forming covalent links with target molecules.

Binding of the probe to a selected support may be accomplished by any of several means. For example, DNA is commonly bound to glass by first silanizing the glass surface, then activating with carbodimide or glutaraldehyde. Alternative procedures may use reagents such as 3-glycidoxypropyltrimethoxysilane (GOP) or aminopropyltrimethoxysilane (APTS) with DNA linked via amino linkers incorporated either at the 3′ or 5′ end of the molecule during DNA synthesis. DNA may be bound directly to membranes using ultraviolet radiation. With nitrocellose membranes, the DNA probes are spotted onto the membranes. A UV light source (Stratalinker™, Stratagene, La Jolla, Calif.) is used to irradiate DNA spots and induce cross-linking. An alternative method for cross-linking involves baking the spotted membranes at 80° C. for two hours in vacuum.

Specific DNA probes may first be immobilized onto a membrane and then attached to a membrane in contact with a transducer detection surface. This method avoids binding the probe onto the transducer and may be desirable for large-scale production. Membranes particularly suitable for this application include nitrocellulose membrane (e.g., from BioRad, Hercules, Calif.) or polyvinylidene difluoride (PVDF) (BioRad, Hercules, Calif.) or nylon membrane (Zeta-Probe, BioRad) or polystyrene base substrates (DNA.BIND™ Costar, Cambridge, Mass.).

B. Nucleic Acid Amplification Procedures

A useful technique in working with nucleic acids involves amplification. Amplifications are usually template-dependent, meaning that they rely on the existence of a template strand to make additional copies of the template. Primers, short nucleic acids that are capable of priming the synthesis of a nascent nucleic acid in a template-dependent process, are hybridized to the template strand. Typically, primers are from ten to thirty base pairs in length, but longer sequences can be employed. Primers may be provided in double-stranded and/or single-stranded form, although the single-stranded form generally is preferred.

Often, pairs of primers are designed to selectively hybridize to distinct regions of a template nucleic acid, and are contacted with the template DNA under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

PCR: A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety. In PCR™, pairs of primers that selectively hybridize to nucleic acids are used under conditions that permit selective hybridization. The term primer, as used herein, encompasses any nucleic acid that is capable of priming the synthesis of a nascent nucleic acid in a template-dependent process. Primers may be provided in double-stranded or single-stranded form, although the single-stranded form is preferred.

The primers are used in any one of a number of template dependent processes to amplify the target gene sequences present in a given template sample. One of the best known amplification methods is PCR™ which is described in detail in U.S. Pat. Nos. 4,683,195, 4,683,202 and 4,800,159, each incorporated herein by reference.

In PCR™, two primer sequences are prepared which are complementary to regions on opposite complementary strands of the target-gene(s) sequence. The primers will hybridize to form a nucleic-acid:primer complex if the target-gene(s) sequence is present in a sample. An excess of deoxyribonucleoside triphosphates is added to a reaction mixture along with a DNA polymerase, e.g., Taq polymerase that facilitates template-dependent nucleic acid synthesis.

If the target-gene(s) sequence:primer complex has been formed, the polymerase will cause the primers to be extended along the target-gene(s) sequence by adding on nucleotides. By raising and lowering the temperature of the reaction mixture, the extended primers will dissociate from the target-gene(s) to form reaction products, excess primers will bind to the target-gene(s) and to the reaction products and the process is repeated. These multiple rounds of amplification, referred to as “cycles,” are conducted until a sufficient amount of amplification product is produced.

A reverse transcriptase PCR™ amplification procedure may be performed in order to quantify the amount of mRNA amplified. Methods of reverse transcribing RNA into cDNA are well known and described in Sambrook et al. (2001). Alternative methods for reverse transcription utilize thermostable DNA polymerases. These methods are described in WO 90/07641, filed Dec. 21, 1990.

LCR: Another method for amplification is the ligase chain reaction (“LCR”), disclosed in European Patent Application No. 320,308, incorporated herein by reference. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750, incorporated herein by reference, describes a method similar to LCR for binding probe pairs to a target sequence.

Qbeta Replicase: Qbeta Replicase, described in PCT Patent Application No. PCT/US87/00880, also may be used as still another amplification method in the present invention. In this method, a replicative sequence of RNA, which has a region complementary to that of a target, is added to a sample in the presence of an RNA polymerase. The polymerase will copy the replicative sequence, which can then be detected.

Isothermal Amplification: An isothermal amplification method, in which restriction endonucleases and ligases are used to achieve the amplification of target molecules that contain nucleotide 5′-[α-thio]-triphosphates in one strand of a restriction site also may be useful in the amplification of nucleic acids in the present invention. Such an amplification method is described by Walker et al. (1992), incorporated herein by reference.

Strand Displacement Amplification: Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA.

Cyclic Probe Reaction: Target specific sequences can also be detected using a cyclic probe reaction (CPR). In CPR, a probe having 3′ and 5′ sequences of non-specific DNA and a middle sequence of specific RNA is hybridized to DNA, which is present in a sample. Upon hybridization, the reaction is treated with RNase H, and the products of the probe identified as distinctive products, which are released after digestion. The original template is annealed to another cycling probe and the reaction is repeated.

Transcription-Based Amplification: Other nucleic acid amplification procedures include transcription-based amplification systems (TAS), including nucleic acid sequence based amplification (NASBA) and 3SR, Kwoh et al. (1989); PCT Application WO 88/10315 (each incorporated herein by reference).

In NASBA, the nucleic acids can be prepared for amplification by standard phenol/chloroform extraction, heat denaturation of a clinical sample, treatment with lysis buffer and mini-spin columns for isolation of DNA and RNA or guanidinium chloride extraction of RNA. These amplification techniques involve annealing a primer, which has target specific sequences. Following polymerization, DNA/RNA hybrids are digested with RNase H while double-stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once against with a polymerase such as T7 or SP6. The resulting products, whether truncated or complete, indicate target specific sequences.

Other Amplification Methods: Other amplification methods, as described in British Patent Application No. GB 2,202,328, and in PCT Application No. PCT/US89/01025, each incorporated herein by reference, may be used in accordance with the present invention. In the former application, “modified” primers are used in a PCR™ like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Davey et al., European Patent Application No. 329 822 (incorporated herein by reference) disclose a nucleic acid amplification process involving cyclically synthesizing single-stranded RNA (“ssRNA”), ssDNA, and double-stranded DNA (dsDNA), which may be used in accordance with the present invention.

The ssRNA is a first template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a second template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Patent Application WO 89/06700 (incorporated herein by reference) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts.

Other suitable amplification methods include “race” and “one-sided PCR™” (Frohman, 1990; Ohara et al., 1989, each herein incorporated by reference). Methods based on ligation of two (or more) oligonucleotides in the presence of nucleic acid having the sequence of the resulting “di-oligonucleotide,” thereby amplifying the di-oligonucleotide, also may be used in the amplification step of the present invention (Wu et al., 1989, incorporated herein by reference).

C. Methods for Nucleic Acid Separation

It may be desirable to separate nucleic acid products from other materials, such as template and excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized with light exhibiting the appropriate excitatory spectra.

### V. PERSONAL HISTORY MEASURES

In addition to use of the genetic analysis disclosed herein, the present invention makes use of additional factors in gauging an individual's risk for developing cancer. In particular, one will examine multiple factors including age, ethnicity, reproductive history, menstruation history, use of oral contraceptives, body mass index, alcohol consumption history, smoking history, exercise history, and diet to improve the predictive accuracy of the present methods. In addition, previous medical findings of atypical ductal hyperplasia or lobular carcinoma in situ contribute to determining a woman's risk of developing breast cancer. A history of cancer in a relative, and the age at which the relative was diagnosed with cancer, are also important personal history measures. The inclusion of personal history measures with genetic data in an analysis to predict a phenotype, cancer in this case, is grounded in the realization that almost all phenotypes are derived from a dynamic interaction between an individual's genes and the environment in which these genes act. For example, fair skin may predispose an individual to melanoma but only if the individual is exposed to prolonged unshielded exposure to the sun's ultraviolet radiation. The inventors include personal history measures in their analysis because they are possible modifiers of the penetrance of the cancer phenotype for any genotype examined. Those skilled in the art will realize that the personal history measures listed in this paragraph are unlikely to be the only such environmental factors that affect the penetrance of the cancer phenotype.

Of particular relevance in applying the methods of the present invention is age stratification. After integrating all age-specific risks, the OncoVue® test report produces the composite estimated risks for an individual for the next 5 years, in age-specific 15, 10 and 15 year intervals respectively (30-44, 45-54, 55-69), and in the remaining lifetime commencing from the patient's current age. These age grouping are utilized to provide accumulated risk over these three periods based upon feedback from clinicians who perform and utilize breast cancer risk assessment tools. However, it is important to point out that OncoVue® risks can also be cumulatively calculated for other age ranges if so desired.

### VI. KITS

The present invention also contemplates the preparation of kits for use in accordance with the present invention. Suitable kits include various reagents for use in accordance with the present invention in suitable containers and packaging materials, including tubes, vials, and shrink-wrapped and blow-molded packages.

Materials suitable for inclusion in a kit in accordance with the present invention comprise one or more of the following:
gene specific PCR primer pairs (oligonucleotides) that anneal to DNA or cDNA sequence domains that flank the genetic polymorphisms of interest;
reagents capable of amplifying a specific sequence domain in either genomic DNA or cDNA without the requirement of performing PCR;
reagents required to discriminate between the various possible alleles in the sequence domains amplified by PCR or non-PCR amplification (e.g., restriction endonucleases, oligonucleotides that anneal preferentially to one allele of the polymorphism, including those modified to contain enzymes or fluorescent chemical groups that amplify the signal from the oligonucleotide and make discrimination of alleles most robust);
reagents required to physically separate products derived from the various alleles (e.g., agarose or polyacrylamide and a buffer to be used in electrophoresis, HPLC columns, SSCP gels, formamide gels or a matrix support for MALDI-TOF).

### VII. CANCER PROPHYLAXIS

In one aspect of the invention, there is an improved ability to identify candidates for prophylactic cancer treatments due to being identified as at a high genetic risk of developing breast cancer. The primary drugs for use in breast cancer prophylaxis are tamoxifen and raloxifene, discussed further below. However, those skilled in the art will realize that there are other chemopreventative drugs currently under development. The disclosed invention is expected to facilitate more appropriate and effective application of these new drugs also when and if they become commercially available.

A. Tamoxifen

Tamoxifen (NOLVADEX®) a nonsteroidal anti-estrogen, is provided as tamoxifen citrate. Tamoxifen citrate tablets are available as 10 mg or 20 mg tablets. Each 10 mg tablet contains 15.2 mg of tamoxifen citrate, which is equivalent to 10 mg of tamoxifen. Inactive ingredients include carboxymethylcellulose calcium, magnesium stearate, mannitol and starch. Tamoxifen citrate is the trans-isomer of a triphenylethylene derivative. The chemical name is (Z)₂-[4-(1,2-diphenyl-1-butenyl) phenoxy]-N,N-dimethylethanamine 2-hydroxy-1,2,3-propanetricarboxylate (1:1). Tamoxifen citrate has a molecular weight of 563.62, the pKa′ is 8.85, the equilibrium solubility in water at 37° C. is 0.5 mg/mL and in 0.02 N HCl at 37° C., it is 0.2 mg/mL.

Tamoxifen citrate has potent antiestrogenic properties in animal test systems. While the precise mechanism of action is unknown, the antiestrogenic effects may be related to its ability to compete with estrogen for binding sites in target tissues such as breast. Tamoxifen inhibits the induction of rat mammary carcinoma induced by dimethylbenzanthracene (DMBA) and causes the regression of DMBA-induced tumors in situ in rats. In this model, tamoxifen appears to exert its anti-tumor effects by binding the estrogen receptors.

Tamoxifen is extensively metabolized after oral administration. Studies in women receiving 20 mg of radiolabeled (¹⁴C) tamoxifen have shown that approximately 65% of the administered dose is excreted from the body over a period of 2 weeks (mostly by fecal route). N-desmethyl tamoxifen is the major metabolite found in patients' plasma. The biological activity of N-desmethyl tamoxifen appears to be similar to that of tamoxifen. 4-hydroxytamoxifen, as well as a side chain primary alcohol derivative of tamoxifen, have been identified as minor metabolites in plasma.

Following a single oral dose of 20 mg, an average peak plasma concentration of 40 ng/mL (range 35 to 45 ng/mL) occurred approximately 5 hours after dosing. The decline in plasma concentrations of tamoxifen is biphasic, with a terminal elimination half-life of about 5 to 7 days. The average peak plasma concentration of N-desmethyl tamoxifen is 15 ng/mL (range 10 to 20 ng/mL). Chronic administration of 10 mg tamoxifen given twice daily for 3 months to patients results in average steady-state plasma concentrations of 120 ng/mL (range 67-183 ng/mL) for tamoxifen and 336 ng/mL (range 148-654 ng/mL) for N-desmethyl tamoxifen. The average steady-state plasma concentrations of tamoxifen and N-desmethyl tamoxifen after administration of 20 mg tamoxifen once daily for 3 months are 122 ng/mL (range 71-183 ng/mL) and 353 ng/mL (range 152-706 ng/mL), respectively. After initiation of therapy, steady state concentrations for tamoxifen are achieved in about 4 weeks and steady state concentrations for N-desmethyl tamoxifen are achieved in about 8 weeks, suggesting a half-life of approximately 14 days for this metabolite.

For patients with breast cancer, the recommended daily dose is 20-40 mg. Dosages greater than 20 mg per day should be given in divided doses (morning and evening). Prophylactic doses may be lower, however.

B. Raloxifene

Raloxifene hydrochloride (EVISTA®) is a selective estrogen receptor modulator (SERM) that belongs to the benzothiophene class of compounds. The chemical designation is methanone, [6-hydroxy-2-(4-hydroxyphenyl)benzo[b]thien-3-yl]-[4-[2-(1-piperidinyl)ethoxy]phenyl]-hydrochloride. Raloxifene hydrochloride (HCl) has the empirical formula C₂₈H₂₇NO₄S.HCl, which corresponds to a molecular weight of 510.05. Raloxifene HCl is an off-white to pale-yellow solid that is very slightly soluble in water.

Raloxifene HCl is supplied in a tablet dosage form for oral administration. Each tablet contains 60 mg of raloxifene HCl, which is the molar equivalent of 55.71 mg of free base. Inactive ingredients include anhydrous lactose, camuba wax, crospovidone, FD& C Blue No. 2 aluminum lake, hydroxypropyl methylcellulose, lactose monohydrate, magnesium stearate, modified pharmaceutical glaze, polyethylene glycol, polysorbate 80, povidone, propylene glycol, and titanium dioxide.

Raloxifene's biological actions, like those of estrogen, are mediated through binding to estrogen receptors. Preclinical data demonstrate that raloxifene is an estrogen antagonist in uterine and breast tissues. Preliminary clinical data (through 30 months) suggest EVISTA® lacks estrogen-like effects on uterus and breast tissue.

Raloxifene is absorbed rapidly after oral administration. Approximately 60% of an oral dose is absorbed, but presystemic glucuronide conjugation is extensive. Absolute bioavailability of raloxifene is 2.0%. The time to reach average maximum plasma concentration and bioavailability are functions of systemic interconversion and enterohepatic cycling of raloxifene and its glucuronide metabolites.

Following oral administration of single doses ranging from 30 to 150 mg of raloxifene HCl, the apparent volume of distribution is 2.348 L/kg and is not dose dependent. Biotransformation and disposition of raloxifene in humans have been determined following oral administration of ¹⁴C-labeled raloxifene. Raloxifene undergoes extensive first-pass metabolism to the glucuronide conjugates: raloxifene-4′-glucuronide, raloxifene-6-glucuronide, and raloxifene-6,4′-diglucuronide. No other metabolites have been detected, providing strong evidence that raloxifene is not metabolized by cytochrome P450 pathways. Unconjugated raloxifene comprises less than 1% of the total radiolabeled material in plasma. The terminal log-linear portions of the plasma concentration curves for raloxifene and the glucuronides are generally parallel. This is consistent with interconversion of raloxifene and the glucuronide metabolites.

Following intravenous administration, raloxifene is cleared at a rate approximating hepatic blood flow. Apparent oral clearance is 44.1 L/kg per hour. Raloxifene and its glucuronide conjugates are interconverted by reversible systemic metabolism and enterohepatic cycling, thereby prolonging its plasma elimination half-life to 27.7 hours after oral dosing. Results from single oral doses of raloxifene predict multiple-dose pharmacokinetics. Following chronic dosing, clearance ranges from 40 to 60 L/kg per hour. Increasing doses of raloxifene HCl (ranging from 30 to 150 mg) result in slightly less than a proportional increase in the area under the plasma time concentration curve (AUC). Raloxifene is primarily excreted in feces, and less than 0.2% is excreted unchanged in urine. Less than 6% of the raloxifene dose is eliminated in urine as glucuronide conjugates.

The recommended dosage is one 60 mg tablet daily, which may be administered any time of day without regard to meals. Supplemental calcium is recommended if dietary intake is inadequate.

C. STAR

More than 400 centers across the U.S., Canada and Puerto Rico are currently participating in a clinical trial for tamoxifen and raloxifene, known as STAR. It is one of the largest breast cancer prevention trials ever undertaken. STAR is also the first trial to compare a drug proven to reduce the chance of developing breast cancer with another drug that has the potential to reduce breast cancer risk. All participants receive one or the other drug for five years. At least 22,000 postmenopausal women at high-risk of breast cancer will participate in STAR. All races and ethnic groups are encouraged to participate in STAR.

Tamoxifen (NOLVADEX®) was proven in the Breast Cancer Prevention Trial to reduce breast cancer incidence by 49 percent in women at increased risk of the disease. The U.S. Food and Drug Administration (FDA) approved the use of tamoxifen to reduce the incidence of breast cancer in women at increased risk of the disease in October 1998. Tamoxifen has been approved by the FDA to treat women with breast cancer for more than 20 years and has been in clinical trials for about 30 years.

Raloxifene (trade name EVISTA®) was shown to reduce the incidence of breast cancer in a large study of its use to prevent and treat osteoporosis. This drug was approved by the FDA to prevent osteoporosis in postmenopausal women in December 1997 and has been under study for about five years.

The study is a randomized double-blinded clinical trial to compare the effectiveness of raloxifene with that of tamoxifen in preventing breast cancer in postmenopausal women. Women must be at least 35 years old, have gone no more than one year since undergoing mammography with no evidence of cancer, have no previous mastectomy to prevent breast cancer, have no previous invasive breast cancer or intraductal carcinoma in situ, have not had hormone therapy in at least three months, and have no previous radiation therapy to the breast.

Patients were randomly assigned to one of two groups. Patients in group one received raloxifene plus a placebo by mouth once a day. Patients in group two received tamoxifen plus a placebo by mouth once a day. Treatment will continue for 5 years.

Quality of life will be assessed at the beginning of the study and then every 6 months for 5 years. Patients will then receive follow-up evaluations once a year. The STAR trial study results were recently released and a 50% reduction in invasive breast cancer incidence was observed for both raloxifene and tamoxifen (world-wide-web at cancer.gov/star).

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Methods

Study Description: OncoVue® was developed from research done on an analysis of SNP genotype variants and clinical/personal history information collected in a decade-long case-control study initiated at the Oklahoma Medical Research Foundation and the University of Oklahoma College Of Medicine and completed at InterGenetics Incorporated. This study included women enrolled in six geographically distinct regions of the U.S. Approximately half were enrolled in the greater Oklahoma City (OK) area from 1996-2006 while the remainder was recruited from Seattle (WA), Southern California (CA), Kansas City (KS/MO), Florida (FL) and South Carolina (SC) from 2003-2006. At all enrollment sites, potential participants were approached consecutively without prior knowledge of disease status. The majority of the participants were enrolled as they presented for appointments at mammography centers. Enrollment in mammography clinics yielded newly diagnosed cases, follow-up cases and cancer-free controls undergoing annual screening. Cases were also enrolled in oncology clinics and controls were obtained in general practice clinics in the same medical complex. Cases and controls were also enrolled at Komen Races or other community-based events. At all collection sites, the majority of individuals approached enrolled in the study. No exclusions were in effect for enrollment in the study. The individuals enrolled in these studies reflect the intended use population for OncoVue®.

Cases were defined as women with a self-reported diagnosis of breast cancer while controls had never been diagnosed with any cancer. All participants were enrolled under informed consent, completed a questionnaire on personal medical history and family history of cancer and provided a buccal cell sample collected in commercial mouthwash. All study protocols were IRB approved, monitored, and performed as previously described (Aston et al., 2005; Ralph et al., 2007).

Datasets: Model development and validation was performed using a dataset of participants ranging in age from 30-69 years with age at diagnosis used for cases and age at enrollment used for controls. The inventors selected the inclusive ages for OncoVue® development and validation to be 30-69 years because of the low number of cases under age 30 and low number of any participants over age 70 enrolled in these studies. In an effort to minimize the potential for confounding factors attributed to ethnicity in the identification of breast cancer risk, initially OncoVue® was developed in a large training set of Caucasian women and tested in another ethnically different population. This is identical to the approach that was taken during the development of the NCI Breast Cancer Risk Model also known as the Gail Model (Gail et al., 1989).

The entire dataset of Caucasian participants was randomly assigned into a “training” set consisting of 80% of all cases and controls. The remaining 20% of Caucasian cases and controls was reserved as an independent “test” set to analyze the performance of the final model built in the training set. The training set consisted of 5,022 women (1,671 BC cases/3,351 cancer-free controls) age-matched to the cases within one year. Age matching was done in an effort to adjust for potential confounding effects due to age-related risk factors when assessing risk factors across different ages. Two independent test sets were utilized to investigate the performance of the final model. The initial test set consisted of 1193 Caucasian women (400 cases and 793 controls). The second test set was an ethnically distinct study of 506 African-American women (142 cases and 364 controls).

Gene polymorphisms: DNAs from the entire sample set were genotyped for 117 common, functional polymorphisms selected from 87 distinct candidate genes (Table 1). Candidate SNPs were selected by criteria that favored those SNPs having a functionally demonstrated and/or predicted physiological consequence as a result of non-synonymous amino acid substitutions, alterations in enzymatic activity or alterations in mRNA transcription rates or stability. Several criteria were utilized for the selection of candidate genes: (1) either known to, or likely to, alter functional activity of the gene or the protein encoded by the gene (most of these polymorphisms have been directly associated with enzymatic and/or physiological alterations and, thus, are not likely to be simply markers in linkage disequilibrium with the causative polymorphisms); (2) demonstrated role in major pathways that influence breast or other cancer development; (3) previously described to be associated with increased or decreased risk of breast and/or other cancers; (4) reasonable allele frequency in the general population.

Genotyping: Genomic DNA was isolated using the Gentra PureGene™ DNA purification kit (Gentra, Minneapolis, Minn.) and stored frozen (−80° C.). Purified genomic DNA was amplified by multiplex PCR performed in an Eppendorf Mastercycler using HotStarTaq™ DNA polymerase (QIAGEN, Inc. Valencia, Calif.). Annealing and extension temperatures were optimized for each multiplex primer set. The primer sequences and specific genotyping conditions are available from the inventors upon request. All of the genotyping assays are currently performed using microbead-based allele-specific primer extension (ASPE) followed by analysis on the Luminex 1100™ (Luminex, Inc. Austin, Tex.). All ASPE assays had reproducibility rates >99.4%. Over 90% of the samples were genotyped using the Luminex technology; some samples were genotyped by PCR-RFLP for some of the variants. The RFLP assays had reproducibility rates of >98%. For all assays, 5% or more of the specimens were genotyped more than once to confirm the internal reproducibility. During all genotyping, operators were blinded to the case-control status of the specimen.

Statistical Analyses: A full range of analyses were performed on both genetic and clinical data, including testing Hardy-Weinberg equilibrium, multivariate logistic regression analysis, evaluation of attributable risks, and estimation of predictive probability.

Characteristics of the Study Population: The genotype frequencies in the general population at steady state are expected to be in Hardy Weinberg Equilibrium (HWE). As a quality control measure, prior to using a genetic polymorphism in model building, the inventors tested the genotypes of controls for HWE. For any given gene, the observed genotype frequencies (f₀, f₁, f₂) were determined for the common homozygotes, heterozygotes, and rare homozygotes in the control dataset. The allelic frequencies were computed from these genotype frequencies and compared to expected frequencies under HWE. The goodness-of-fit χ² test was used to determine if the observed genotype frequencies deviate from those expected under HWE (Hartl and Clark, 1997). All of the 117 SNPs used in the candidate panel conformed to HWE (p>0.05) in the control population and were used in subsequent model building analyses.

Furthermore, in both the training and test sets, the observed genotype frequencies conform to the expectations under HWE at a p-value cut off of 0.05 in the age group in which each SNP is utilized in OncoVue®. Conformation to HWE expectations is commonly utilized to monitor data quality control for several reasons. First, HWE of controls provides assurance of robust and accurate genotyping of the SNPs. Systematic errors in genotyping accuracy frequently manifest as departures of the observed genotype frequencies from HWE in controls. Second, departure from HWE can be indicative of a recent mixing of two or more previously distinct populations. Such recent population mixing can increase the possibility that population stratification issues are distorting the observed associations with breast cancer risk. Conformation that the genotype frequencies are in HWE supports the contention that the controls are being drawn from a homogeneous population and decreases the possibility that population stratification issues have resulted in false discovery of informative SNPs included in the OncoVue® test.

Model Building: An important feature of OncoVue® was the selection of relevant SNPs that added discriminatory accuracy to the final predictive models without being penalized excessively by multiple comparisons. Towards this objective, the inventors used the following model building strategy and validation. First, the entire data set was randomly assigned into a training set consisting of 80% of all cases and controls. The remaining 20% of cases and controls were reserved for use as a validation data set to test “frozen” models built in the training set. The primary analytical goal of the training process was to systematically evaluate genotypic and personal history associations with case-control status using multivariate logistic regression modeling (Hosmer and Lemeshow, 2000).

Penetrance for certain SNPs are strongly age-dependent (i.e., penetrance of a SNP can be appreciable at certain ages, but reduced at other ages (Ralph et al., 2007) the modeling analyses utilized multivariate logistic regression and evaluated terms in both age invariant and age interactive manner for their contribution to risk prediction. Analyses of the case-control training set were performed to identify informative and stable terms as follows: (1) the top 25% of SNPs based on a univariate χ² p-value were selected; (2) the reduced dataset was modeled with a forward stepwise selection method and subjected to 5000 bootstrap resamples to calculate standard error (Efron and Gong, 1983) using a selection p-value of 0.1 and the exit p-value of 0.05. The maximum number of steps allowed was 100.

These iterative analyses were initially performed on the model building dataset to identify informative terms for ages 30 through 69. Published analyses of several candidate SNPs have demonstrated both “pre-” and “post-” menopause specific associations when stratified at age 50 or by first-degree relative status (Thompson et al., 1998; Wedren et al., 2003; Bergman-Jungestrom et al., 1999; De Vivo et al., 2004; Jupe et al., 2001; Nelson et al., 2005; Zhu et al., 2005). To capture these complexities, age strata were analyzed based on presence or absence of at least one first-degree relative with breast cancer. To keep our models parsimonious, informative terms identified for ages 30-69 were not included as candidate terms in subsequent analyses. Analyses of the age 50-69 group did not identify additional informative terms. The informative terms identified overall (30-69) and within each strata of 30-49 year olds (by family history) were combined into a single MLR. The inventors utilized maximum likelihood estimates to produce a single integrated multifactorial risk estimator (MFRE) which then computed an individualized relative risk associated with the disease state.

Finally, the informative terms identified in each of these model building analyses were combined and characterized in the static training set without additional bootstrapping.

Model Validation: The final predictive model produced from analysis of the training data set was “frozen” and the performance characteristics were tested in two independent validation data sets. The first set of samples consisted of 20% of the Caucasian women that were not a part of the training set used in the model building process. The second was an additional independent validation set of African American cases and controls collected in InterGenetics overall studies. The validation strategy and performance of the OncoVue® model was evaluated by comparison to the performance of the Gail model.

### Example 2

### Results

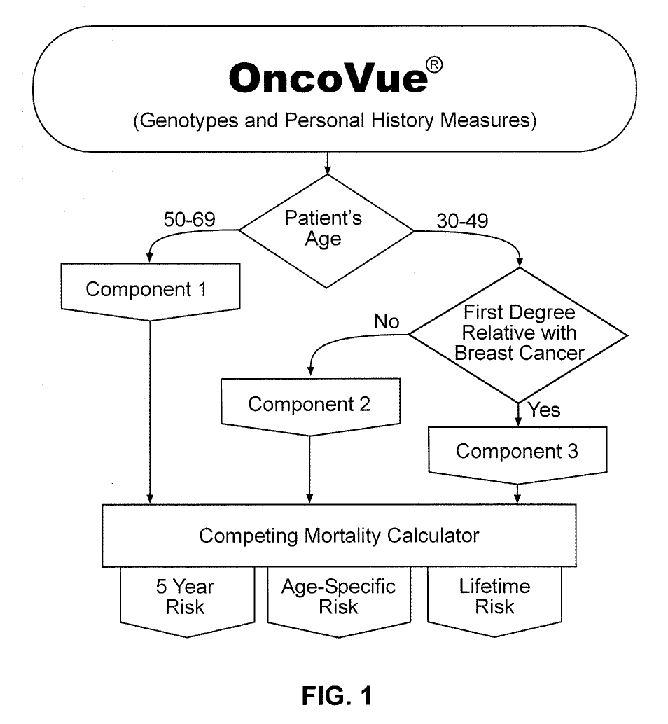

Algorithm Architecture and Implementation: The OncoVue® test is a tri-partite model built of three integrated components derived from multivariate logistic regression analyses on input data containing 117 genetic polymorphisms, 7 individual personal history measures, and the composite Gail model score. Because breast cancer is a complex disease and may arise through multiple etiologies, the OncoVue® model was developed with this in mind. The model was built incrementally from the analysis of a training set consisting of 1671 breast cancer cases and 3351 cancer-free controls age-matched to the cases within one year. FIG. 1 shows an overview of the components that make up the OncoVue® algorithm, starting with the patient's current age and history of a first degree relative with breast cancer and Table 2 shows the terms and parameter estimates of the different components of OncoVue®. Each component of the model evaluates SNPs and personal history measures individually and interacting with age to calculate individualized risks for the patient.

The predictive model includes three stratified multivariate logistic regression (MLR) components (Component 1: SNPs and PHMs identified for women 50-69 years, Component 2: SNPs and PHMs identified for women 30-49 years without family history, and Component 3: SNPs and PHMs identified for women 30-49 years with family history). Each regression component includes a subset of predictive genetic markers specific to the corresponding age strata.

All three model components presented in Table 2 represent up to three composite SNP and PHM models—Composite model 1, 2, and 3. Each of these three composite models is a multivariate model that produces a log odds of developing breast cancer, similar to a Gail Score. The three composite models are layered upon one another through MLR resulting in components 1, 2, and 3. The components presented are a result of the following composite models:
Component 1=CM 1
Component 2=0.67358+1.03389*CM1+0.90304*CM2
Component 3=1.5784+0.9104*CM1+1.2463*CM2+1.01934*CM3
   where CM1, CM2, and CM3 represent the three composite models.

Component 1 contains a “Number of Relatives” term; therefore, the term is still present in component 2. The term adds no additional odds to the component, since all subjects passing through component 2 have no relatives. Numerically a zero is multiplied times the −1.26 coefficient reported on the table resulting in a zero for all members of this component.

The algorithm estimates an individual's probability of developing breast cancer over time, based upon a set of selected SNPs from multiple genes as well as clinical/personal history measures. The actual algorithm is implemented by an R language script, to facilitate an accurate and reproducible calculation. After integrating all age-specific risks, the OncoVue® test report produces the composite estimated risks for an individual for the next 5 years, in age-specific 15, 10 and 15 year intervals respectively (30-44, 45-54, 55-69), and in the remaining lifetime commencing from the patient's current age. These age grouping are then utilized to provide accumulated risk over these three periods based upon feedback from clinicians who perform and utilize breast cancer risk assessment tools.

OncoVue® produces estimated probabilities of breast cancer risks for 5, 10 and 15 years, respectively (30-44, 45-54, 55-69), and in the remaining lifetime from the patient's current age, based on the following calculations. First, OncoVue® computes individual odds ratios associated with disease state using the three multivariate logistic regression components identified above.

The second step is to compute attributable risks as previously described (Bruzzi et al., 1995). Then, by multiplying their complement to breast cancer incidence, obtained from SEER³, the inventors obtained the baseline hazard rate for breast cancer, denoted as h₁(t,X)=h_{baseline}(t)RR (t,X), where X denotes combined genetic and PHM variables.

The third step is to account for mortality hazard rates, which are obtained from the Census figures utilized in the above cited SEER database and denoted as h₂(t). Then, the probability of being diagnosed with breast cancer for the next τ years, from the current age a is calculated via

$$Pr ( a , τ , X ) = \frac{{\int }_{a}^{a + τ} {h}_{1} ( u , X ) exp [ - {\int }_{0}^{u} \{ {h}_{1} ( t , X ) + {h}_{2} ( t ) \} d t ] d u}{exp [ - {\int }_{0}^{a} \{ {h}_{1} ( t , X ) + {h}_{2} ( t ) \} d t ]} ,$$

where the integration is over the range specified in the integrands (Gail et al., 1989). Using the above formula, those probabilities can be computed for the next 5 years, for any age-specific interval and lifetime starting at the current age. OncoVue® computes and reports risks for three age-specific intervals from 30-44, 45-55 and 55-69, representing what the inventors have designated pre-, peri- and post-menopausal intervals, respectively.

The OncoVue® breast cancer risk model is indexed by Gail score-related clinical/demographic variables and selected SNP genotypes, as well as by corresponding regression coefficients and population-based incidence and mortality rates. In the context of computing individual risk probability, SNP genotypes and clinical variables are known. The population-based incidence rate is extracted from the population-based SEER registry, and is taken to be known and fixed (SEER, 2005; www.seer.cancer.gov). Population-based mortality rates are extracted from the population census, and are taken to be known and fixed (www.cdc.gov/nchs). Estimated regression coefficients in OncoVue® are estimated from our large case-control training set with random variations due to limited sample size of 5000. Hence, the estimated risk probability from our predictive model is associated with random variability. Therefore, from a statistical perspective, it is necessary to compute the confidence interval for each individual estimate of risk probability.

In the literature, Benichou and Gail (1990, 1995) provided methods for computation of variance and confidence interval for estimating risk probability. Their calculation considers two sources of variations: one source, which is the same as ours, arises from estimating odds ratios in a case-control study, and another source is from estimating incidence rates in the follow-up cohort. Because the inventors are not estimating incidence rates in any follow-up cohort, this portion of the calculation is not directly applicable to ours. However, the general principle of constructing confidence intervals remains the same.

Following the statistical principle developed by Benichou and Gail, the inventors use the estimation procedure that produces confidence intervals for estimated risk probability. The OncoVue® report contains three MLR components: Component 1 consists of SNPs and PHMs identified for women 50-69 years, Component 2 consists of SNPs and PHMs identified for women 30-49 years without family history, and Component 3 consists of SNPs and PHMs identified for women 30-49 years with family history. From the application of the model to these components, the inventors estimate their covariance matrices, denoted as Σ_{C1}, Σ_{C2} and Σ_{C3}, respectively, along with their corresponding regression coefficient vectors β_{C1}, β_{C2} and β_{C3}, associating with the same set of clinical variables applied to these specific age groups. When a patient's corresponding clinical variables and SNP genotypes are determined, the inventors compute their log odds ratios: LORⱼ=βⱼ*Xⱼ, where the subscript j corresponds to three component group indicator. The variance is estimated by Vⱼ=(Xⱼ)′*Yⱼ*Xⱼ. Now given the population-based baseline hazard rates (from SEER) in thirteen age intervals h₀(t) (30-, 35-, . . . , 65-69) and also computed attributable risk, the inventors can compute the hazard function for the patient with their clinical and SNP genotypes via

h₁(t,X)=h₀(t)exp[LOR(t,X)], [1]

In which the overall log odds ratio is written as

LOR(t)=LOR(X_{C1})I(50≦t≦69,)+LOR(X_{C2})I(30≦t≦49,no)+LOR(X_{C3})I(30≦t≦49, yes) [2]

where I(t, family history) is the binary indicator function for the corresponding component, t represents age, family history of breast cancer is represented by a yes or no, and a “.” represents not applicable. The estimated risk probability is computed via the following calculation:

$$Pr , ( a , τ , X ) = \frac{{\int }_{a}^{a + τ} {h}_{1} ( u , X ) exp [ - {\int }_{0}^{u} \{ {h}_{1} ( t , X ) + {h}_{2} ( t ) \} d t ] d u}{exp [ - {\int }_{0}^{a} \{ {h}_{1} ( t , X ) + {h}_{2} ( t ) \} d t ]} , \qquad [ 3 ]$$

where a is the current age, τ is the age interval for prediction, and h₂(t) is the mortality rate. Clearly, this risk probability is a non-linear function of log ORⱼ. To improve the numerical properties of the estimated confidence interval, the inventors transform the risk probability via a logistic function:

$$F ( LOR ) = logit [ Pr ( a , τ , X ) ] = log \frac{Pr ( a , τ , X )}{1 - Pr ( a , τ , X )} . \qquad [ 4 ]$$

To compute variance of the above logit probability, the inventors apply the delta-method, which is commonly used to compute variance of non-linear function of estimate (Cox and Hinkley, 1974). Specifically, the variance of the non-linear function F(LOR) can be written as

$$var [ F ( LOR ) ] = {[ \frac{\partial F ( LOR )}{\partial ( LOR )} ]}^{2} var ( LOR ) , \qquad [ 5 ]$$

where

$$\frac{\partial F ( LOR )}{\partial ( LOR )}$$

is the first derivative and var(LOR) is the variance of estimated log odds ratio. Let V_{C1}, V_{C2} and V_{C3} denote variances of log odds ratios for components 1, 2, and 3. Since variances of LOR are estimated separately for each component, the total 5 variance of estimated logit probability may be written as:

var(LOR)=V_{C1}I(50≦t≦69,)+V_{C2}I(30≦t≦49, no)+V_{C3}I(30≦t≦49, yes) [6]

The computation of the first derivative

$$\frac{\partial F ( LOR )}{\partial ( LOR )}$$

is made possible from the chain-rule decomposition. It may be written as

$$\begin{matrix}\frac{\partial F ( LOR )}{\partial ( LOR )} = \frac{\partial F ( LOR )}{\partial Pr ( a , τ , X )} \frac{\partial Pr ( a , τ , X )}{\partial ( LOR )} \\ = {( \frac{\partial Pr ( a , τ , X )}{\partial F ( LOR )} )}^{- 1} \frac{\partial Pr ( a , τ , X )}{\partial ( LOR )}\end{matrix} \qquad [ 7 ]$$

in which the first part equals

$${( \frac{\partial Pr ( a , τ , X )}{\partial ( LOR )} )}^{- 1} = {\{ Pr ( a , τ , X ) ( 1 - Pr ( a , τ , X ) ] \}}^{- 1} . \qquad [ 8 ]$$

The second part simply is the derivative of Pr(a, τ, X) over LOR in h₁(t,X) in equations [1] and [2], except it does not have any simple and explicit representation. Now the inventors can compute the 95% confidence interval for F(LOR) via:

[F(LOR)−1.96*\sqrt{V_{L}},F(LOR)+1.96*\sqrt{V_{L}}], [9]

which should have 5% error rate on two-sided test. Taking the anti-logit transformation, one obtains the desired 95% confidence interval for Pr(a,τ,X).

The computational protocol for computing variance of estimated individual risk probability includes the following steps:

From fitted logistic regression models for three age groups, the inventors obtain covariance matrices Σ_{C1}, Σ_{C2} and Σ_{C3} for their corresponding regression coefficients (i.e., log odds ratios) in different components.

When the subject's genotypes are known and are coded according to covariate coding, the inventors can then compute their corresponding log odds ratios.

LOR_{C1}=\hat{β}′_{C1}X_{C1}

LOR_{C2}=\hat{β}′_{C2}X_{C2}

LOR_{C3}=\hat{β}′_{C3}X_{C3}

where parameters with subscript “young”, “middle” and “old” are estimated from their corresponding age groups, and X with appropriate subscript correspond coding of known genotypes, in addition to clinical variables in the Gail model. These values are used for computing the individual risk probability.

In addition, the inventors compute their variances with known genotypes as

V_{C1}=\hat{β}′_{C1}Σ_{C1}\hat{β}_{C1}

V_{C2}=\hat{β}′_{C2}Σ_{C2}\hat{β}_{C2}

V_{C3}=\hat{β}′_{C3}Σ_{C3}\hat{β}_{C3}
Next, the inventors compute the derivative of F(LOR) with respect to LOR, which does not have any explicit form. In the initial implementation, the inventors will use the numerical approximation, which is computed as the following. Taking Δ=10⁻⁸, the inventorscompute F(LOR+Δ) and F(LOR). The numerical approximation of the first derivative equals

$$\frac{\partial F ( LOR )}{\partial ( LOR )} \approx \frac{F ( LOR + Δ ) - F ( LOR )}{Δ} ,$$

The precision of the above approximation is expected to be sufficiently high.
Finally, the inventors compute the variance of F(LOR) via the equation [4] and 95% C1 via the equation [6].

In total, twenty-two SNPs located in nineteen genes comprise the OncoVue® model. All of these genes are either directly or indirectly involved in various tumorigenesis pathways (Table 3). Seven SNPs are in genes involved in steroid hormone synthesis, signaling or metabolism. A SNP in the vitamin D receptor gene, which shares many features with steroid hormone receptors, is included in OncoVue®. Five SNPs are in genes that are directly involved in various aspects of DNA repair. In addition, three SNPs in the gene encoding acetyl-CoA carboxylase alpha (ACACA) were individually informative and are included in OncoVue®. ACACA is involved in lipid metabolism but also interacts directly with BRCA1 (Magnard et al., 2002 and Sinilnikova et al., 2004), a gene that when mutated causes familial breast and ovarian cancer predisposition syndrome. The remaining selected SNPs were in the genes encoding insulin, insulin-like growth factor 2, microsomal epoxide hydrolase (EPHX1), and the human tissue kallikrein, KLK2.

The goal in the development of OncoVue® was to extend the Gail model to improve estimation of individual risk. The Gail model is the most common clinically utilized predictive model for estimating breast cancer risk in women without exceptional family histories (Gail et al., 1989; Constantino et al., 1999). It utilizes age at first live birth, age at menarche, first-degree family history of breast cancer and history/outcome of benign breast biopsies to estimate individual-level relative risk. Following the incorporation of the population age-specific breast cancer incidence rates, the Gail model reports the probability of being diagnosed with breast cancer in pre-specified windows, such as next five year or lifetime risk. The Gail model has been found to accurately estimate the number of cases that will emerge in specific risk strata but it only exhibited modest discriminatory accuracy for the individual (Rockhill et al., 2001).

The performance characteristics of OncoVue® were examined and compared to the Gail model in the training set and tested in the Caucasian (Test 1) and African American (Test 2) sample sets. The ability of OncoVue® to better identify and classify women that are truly at higher risk for breast cancer (previously diagnosed breast cancer cases) than the Gail model alone was examined in a number of ways as discussed below.

Table 4 shows the results of analyses in which the number and ratio of cases and controls placed at higher risk by OncoVue® compared to Gail was determined using two risk level cut-off thresholds (>2.0% and >3.0%) that approximate clinically moderate and high risk categories in the age groups examined. In addition, the agreement in relationship to the overlap between the individuals placed into each of these risk categories was examined by using the kappa statistic. To parallel stratifications utilized in constructing the model and in the report output, the performance of OncoVue® for individuals in various age groups was examined.

The results show that in the majority of the age categories in both the Training and Test sets, OncoVue® correctly places more cases and fewer controls at high risk compared to the Gail model (O/G ratio>1.0). Because the Gail model exhibits low discriminatory accuracy, it is also important to know that the individuals placed at high risk by OncoVue® are not simply the same individuals placed at high risk by the Gail model. This was examined by calculating the kappa statistic as a measure of the agreement in patient categorization between the two models. For example, in the Training set (30-44) at a risk level of >3%, the kappa of 0.50 shows that 50% of the subjects are categorized identically between the two tests while 50% of the subjects have a different risk classification when OncoVue® is utilized. Across all of the categories, 34% or more of the moderate-high risk individuals are uniquely classified by OncoVue®. Taken together with the improvement in correct classification of cases in the high risk category, these results demonstrate OncoVue® increased predictive accuracy for breast cancer risk in the populations studied. In order to further define the origin of the observed differences and confirm that they do not originate from a classification error, analyses were performed to examine the Concordance Statistic or area under the ROC curve along with the fold-stratification of patients.

Table 5 shows the fold-stratification computed for both the ratio of ranges (high to low) and the ratio of the 95^{th}/5^{th} percentile range for cases and controls. In the breast cancer cases, the OncoVue® fold-stratification exceeded that of the Gail model, with OncoVue® showing greater stratification of risk. At the extremes, OncoVue® shows an almost 6-fold stratification in the Cases from 30-44 and a 4-fold stratification in the Cases from 30-49 in the training set. The 95^{th}-5^{th} percentile analyses also demonstrate the increased ability of OncoVue® to stratify the population compared to the Gail model with a 1.5 to 2-fold stratification of the cases the Training and Test sets in these age groups. In the controls, a 2-fold increased stratification was also observed in some categories. This is not surprising because even though they are controls the general population will have individuals at very high risk of developing breast cancer.

The extended stratification observed particularly for cases for the OncoVue® model provides evidence of an improved ability to spread the risk of breast cancer cases over a greater range compared to the Gail Model. Similarly, the small stratification observed in controls between the 95-5^{th} percentile might be attributed to the fact that controls, in general, have a low risk and the fact that probabilities are bound numerically at zero. To test whether these hypotheses are plausible and determine if OncoVue® doesn't simply exhibit more variability due the large number of additional terms, the inventors examined discriminatory accuracy above random chance for OncoVue® compared to the Gail Model results alone. Table 6 shows the results from these analyses.

The data indicate that in the Training Set OncoVue® outperformed the Gail Model with a statistically significant 17% improvement above random compared to only an 8% improvement for the Gail Model. In Test Set 1, OncoVue® exhibits a statistically significant improvement compared to the Gail Model (14% vs. 7%) and the 95% CI for OncoVue® ranges from 8% to 20% above random chance. Conversely, the Gail model's predictive ability was only 7% and numerically only a marginal improvement over a coin toss with a 95% CI that ranges from 0.8% to 13%.

Table 7 presents the results obtained when the statistical significance of the percent average improvement was tested. These results indicate that in all the Training sets at all age ranges, OncoVue® has statistically significantly better discriminatory accuracy than the Gail model (p<0.0001). For example, the training set for the age range of 30-44 demonstrated an 52% improvement in discriminatory accuracy, with the 95% confidence interval around the improvement of 35% to 70%. The difference in discriminatory accuracy is also validated in Test Set 1 (100% difference, p=0.018). Similar results were obtained in the Training set and Test Set 1 in the 30-49 age group with statistically significant improvement of 50% and 40%, respectively. Overall, the statistically significant improvement in performance of OncoVue® compared to the Gail model alone in Test set 1 demonstrates improved clinical utility for use in the assessment of breast cancer risk. A trend toward increased discriminatory accuracy was noted in Test set 2, the African American cohort for the same age group, a trend toward increased discriminatory accuracy was noted, but the sample set was not large enough to have the power to reach statistical significance.

The likelihood ratio provides an excellent measure of clinical performance and utility because it incorporates both sensitivity and specificity and is not sensitive to population characteristics and disease prevalence (Guyatt and Rennie, 2002; Ebell, 2001). The positive likelihood ratio (PLR) was calculated as the proportion of patients with breast cancer that received an elevated risk estimate divided by the proportion of disease-free individuals with an elevated risk estimate. These analyses used a risk of ≧12% as the cut-off threshold for elevated risk. This represents a 1.5-fold increase over the ˜8% mean risk of controls across the age range from 30-69. The PLR was calculated individually for both OncoVue® and the Gail Model which represents the current clinical standard for breast cancer risk assessment. An improved test would be expected to exhibit an increased PLR. The potential fold-improvement for OncoVue® compared to the Gail Model was calculated by dividing the PLR for OncoVue® by the PLR for the Gail Model. The statistical significance of the calculated fold-improvement was assessed using a χ²-test. Table 8 shows the results of these analyses for the Training Set, Test 1, Test 2 and the Blinded Validation study which was an independently collected sample set analyzed with InterGenetics remaining blinded to case-control status. The Blinded Validation set is an independently collected study conducted by investigators at the University of California San Francisco and the Buck Institute for Age Research that involved analysis of 177 controls and 169 age-matched women diagnosed with breast cancer between 1997 and 1999 that had enrolled in the Marin County, California breast cancer adolescent risk factor study (Clarke et al., 2002; Wrensch et al., 2003). All DNA samples were anonymously coded to remove case-control status and provided to InterGenetics along with all other relevant personal history information. DNAs were genotyped for the 22 SNP variants in OncoVue® and combined with personal factors to calculate the risk scores for the individual participants. OncoVue® scores were then returned to the Marin County study investigators who added case-control status and completed analysis of model performance.

Table 8 shows the PLRs for OncoVue® and the Gail Model as well as the fold-improvement calculated using the risk threshold of ≧12% to define elevated risk. The PLR in the training set was 2.1 with reassuringly similar values in the three independent test or validation sets. Thus, OncoVue® is generalizable to other populations. Similar reproducibility but lower PLRs were obtained for the Gail Model indicating that OncoVue® improves individual risk estimation. Fold-improvements in the PLR of OncoVue® over the Gail Model of 1.8, 1.7, 2.2, and 2.4 respectively for the Training Set and the three validation sets are statistically significant (p<0.0001, p=0.024, p=0.034, and p=0.036). This trend in fold improvement increases at higher cut-off thresholds. For example, at the 20% threshold, the fold improvement in the Training set is 3.0 (p<0.0001) and in validation set 1 is 2.1 (p=0.07), but could not be calculated for validation set 2 or the Marin County study due to lack of controls at this elevated risk level.

Another measure of clinical utility for OncoVue® is the placement of more breast cancer cases at elevated risk compared to a fixed number of controls, when referenced to the Gail Model. Because the distribution of risk estimates assigned by OncoVue® and the Gail Model varies, this was examined by first ranking and counting the number of controls and cases with Gail Model risk scores ≧the 12% risk threshold level. Table 2 shows this analysis of the number of breast cancer cases identified at elevated risk by OncoVue® based upon fixed control levels, as determined from Gail Model risk estimates. Using this number of controls (i.e., 760, 161, 56, and 43 in the Training, Test 1, Test 2 and Blinded Validation sets, respectively) as a reference point for the same number of controls identified by OncoVue®, the number of corresponding breast cancer cases identified by OncoVue® always exceeded the Gail Model. The percent improvement in number of cases identified ranged from 14 to 51%.

Although any single term included in OncoVue® only exhibits a modest association with breast cancer risk, collectively these genetic factors, and additionally considered with personal factors, produce a risk estimator with significantly improved discriminatory accuracy and clinical utility. The improvement in risk estimation by OncoVue®, and the confirmation of this improvement in three independent validation sets, including one ethnically distinct population and a blinded validation using a previously collected sample set, demonstrates the value of this model building approach and its applicability to other complex diseases. SNP genotypes associated with cancer and other complex diseases identified in the large number of GWA studies that have been published have clearly demonstrated that any given SNP variant will only demonstrate modest associations. Thus, an integrated model building approach that attempts to capture the complexity of biological pathways and clinical/personal risk factors in influencing the etiopathogenesis of cancer will produce the most accurate risk assessment tool.

TABLE 1
ALL SNPs EXAMINED

| SNP ID* | GENE SYMBOL | GENE NAME | CHROMOSOME | LOCATION | SNP ALLELES |
|---|---|---|---|---|---|
| NA | ACACA(=ACCa) | acetyl-Coenzyme A carboxylase alpha | 17q21 | 5′UTR | T→C |
| | | | | 5′UTR-86 | |
| NA | ACACA(=ACCa) | acetyl-Coenzyme A carboxylase alpha | 17q21 | pIII | T→G |
| | | | | pIII-724 | |
| NA | ACACA(=ACCa) | acetyl-Coenzyme A carboxylase alpha | 17q21 | IVS8 | T→C |
| | | | | IVS8-16 | |
| NA | ACACA(=ACCa) | acetyl-Coenzyme A carboxylase alpha | 17q21 | IVS17 | T→C |
| | | | | IVS17 + 66 | |
| rs4646994 | ACE16 | Angiotensin I-Cconverting Enzyme | 17q23 | Alu, intron 16 | Ins/Del |
| rs1136410 | ADPRT | ADP-ribosyltransferase (NAD+; poly | 1q42 | Val762Ala | C→T |
| | | (ADP-ribose) polymerase) | | | |
| rs28997576 | BARD1 (C557S) | BRCA1-Associated Ring Domain 1 | 2q34-q35 | Cys557Ser | G→C |
| rs1048108 | BARD1 (P24S) | BRCA1-Associated Ring Domain 1 | 2q34-q35 | Pro24Ser | C→T |
| rs2229571 | BARD1 (R378S) | BRCA1-Associated Ring Domain 1 | 2q34-q35 | Arg378Ser | G→ C |
| NA | BRCA1 | Breast Cancer Protein Type 1 | 17q21 | 3875delGTCT | Wt/Mut |
| NA | BRCA1 | Breast Cancer Protein Type 1 | 17q21 | 4184delTCAA | Wt/Mut |
| rs799917 | BRCA1 | Breast Cancer Protein Type 1 | 17q21 | Pro830Leu | C→T |
| rs1799966 | BRCA1 | Breast Cancer Protein Type 1 | 17q21 | Ser1613Gly | A→G |
| rs206340 | BRCA2 | Breast Cancer Protein Type 2 | 13q12.3 | intron 24 | G→A |
| rs144848 | BRCA2 | Breast Cancer Protein Type 2 | 13q12.3 | Asn372His | C→A |
| rs 603965 | CCND1 | Cyclin D1 (PRAD1: parathyroid | 11q13 | Pro242Pro | G→A |
| | | adenomatosis 1) | | | |
| rs4680 | COMT | Catechol-O-methyltransferase | 22q11.2 | Val158Met | G→A |
| rs5275 | COX2 | Cyclooxygenase 2 | 1q25.2-25.3 | nt8473, 3′UTR | T→C |
| rs4646903 | CYP1A1 | Cytochrome P450 Family 1A, | 15q22-q24 | 3′UTR | T→C |
| | | polypeptide 1 | | | |
| rs1048943 | CYP1A1 | Cytochrome P450 Subfamily 1, | 15q22-24 | Ile462Val | A→G |
| | | polypeptide 1 | | | |
| rs10012 | CYP1B1 (R48G) | Cytochrome P450 SubFamily 1B | 2p22-p21 | Arg48Gly, exon 2 | C→G |
| rs1056836 | CYP1B1 (V432L) | Cytochrome P450, family 1, subfamily B, | 2p22-p21 | Val432Leu | C→G |
| | | polypeptide 1 | | | |
| rs1800440 | CYP1B1 (N453S) | cytochrome P450, family 1, subfamily B, | 2p22-p21 | Asn453Ser | A→G |
| | | polypeptide 1 | | | |
| rs1799998 | CYP11B2 | Cytochrome P450 Family XIB | 8q21 | promoter, nt-344 | C→T |
| | | polypeptide 2 | | | |
| rs743572 | CYP17 | Cytochrome P450, family 17, subfamily | 10q24.3 | 5′UTR | T→C |
| | | A, polypeptide 1 | | | |
| rs10046 | CYP19 (E10) | Cytochrome P450 Family 19 | 15q21.1 | 3′UTR, exon 10 | T→C |
| rs700519 | CYP19 (R264C) | Cytochrome P450 Family 19 | 15q21.1 | Arg 264Cys, Exon 8 | C→T |
| rs16947 | CYP2D6 | Cytochrome P450, Subfamily IID, | 22q13.1 | Arg296Cys | C→T |
| | | polypeptide 6 | | | |
| rs16260 | ECAD | E-Cadherin | 16q22.1 | promoter, nt-160 | A→C |
| rs4444903 | EGF | Epidermal growth factor (beta- | 4q25 | 5′UTR, nt61 | G→A |
| | | urogastrone) | | | |
| rs1051740 | EPHX1 | Epoxide hydrolase (microsomal) | 1q42.1 | Tyr113His, exon 3 | T→C |
| rs2077647 | ESR1 (=ERA) | Estrogen Receptor α | 6q25.1 | codon 10 neutral | T→C |
| rs3212986 | ERCC1 | Excision repair cross-complementing | 19q13.2-q13.3 | 3′UTR (nt 8092) | C→A |
| | | rodent | | | |
| | | repair deficiency, complementation group 1 | | | |
| rs1052559 | ERCC2 | Excision repair cross-complementing | 19q13.3 | Lys751Gln | A→C |
| | (=XPD) | rodent repair deficiency, | | | |
| | | complementation group 2(xeroderma | | | |
| | | pigmentosum D) | | | |
| rs1800067 | ERCC4 (=XPF, RAD1) | Excision repair cross-complementing | 16p13.3-p13.11 | Arg415Gln | G→A |
| | | rodent | | | |
| | | repair deficiency, complementation group 4 | | | |
| Rs1800682 | FAS (TNFRSF6) | Tumor Necrosis Receptor Superfamily | 10q24.1 | promoter, nt-670 | G→A |
| | | member 6 | | | |
| rs763110 | TNFSF6 (=FASL) | FAS Ligand | 1q23 | 5′ promoter, nt-844 | T→C |
| rs351855 | FGFR4 | Fibroblast Growth Factor 4 | 5q35.1-qter | Gly388Arg | G→A |
| rs681673 | GADD45 | Growth Arrest and DNA-Damage | 1p34-p12 | intron 3, nt 2441 | T→C |
| | | Inducible Gene 45 alpha | | | |
| NA | GSTM1 | Glutathione S-transferase (m family) | 1p13.3 | gene deletion (16 kb) | +/− |
| rs947894 | GSTP1 | Glutathione S-transferase pi | 11q13 | Ile105Val | G→A |
| rs1136201 | HER2 (=ERBB2) | v-erb-b2, erythroblastic leukemia viral | 17q21.1 | Ile655Val | A→G |
| OR | | oncongene | | | |
| rs1801200 | | | | | |
| rs1801201 | HER2 (=ERBB2) | v-erb-b2, erythroblastic leukemia viral | 17q21.1 | Ile654Val | A→G |
| | | oncongene | | | |
| rs1058808 | HER2 (=ERBB2) | v-erb-b2, erythroblastic leukemia viral | 17q21.1 | Ala1170Pro | G→C |
| | | oncongene | | | |
| rs1800562 | HLA-H (=HFE) | Hereditary Haemochromatosis Gene | 6p21.3 | Cys282Tyr | G→A |
| rs1799945 | HLA-H (=HFE) | Hereditary Haemochromatosis Gene | 6p21.3 | His63Asp | C→G |
| rs12628 | HRAS | Harvey rat sarcoma viral oncogene | 11p15.5 | nt81 codon 27, | T→C |
| | | homolog | | neutral | |
| rs5498 | ICAM1 | Intercellular Adhesion Molecule 1 | 19p13.3-p13.2 | Lys469Glu | A→G |
| rs1056538 | ICAM5 | Intercellular Adhesion Molecule 5 | 19p13.2 | Val301Ile | G→A |
| rs2000993 | IGF2 | Insulin like Growth Factor 2 | 11p15.5 | nt 3580 | G→A |
| rs1800795 | IL6 | Interleukin 6 | 7p21 | promoter nt-174 | G→C |
| rs1800896 | IL-10 | Interleukin 10 | 1q31-q32 | Nt-1082, promoter | A→G |
| rs3842752 | INS | Insulin | 11p15.5 | nt1107 | C→T |
| rs5918 | ITGB3 | Integrin β3 | 17q21.32 | Leu33Pro | T→C |
| rs198977 | KLK2 | Kallikrein 2 | 19q13 | Arg226Trp | C→T |
| rs3745535 | KLK10 | Kallikrein 10 | 19q13.33 | Ala50Ser | G→T |
| rs1799986 | LRP1 | Low density lipoprotein receptor related | 12q13-1-q13.3 | Cys766Thr | C→T |
| | | protein 1 | | | |
| rs2279744 | MDM2 | Mouse double minute 2 homolog | 12q14-q15 | promoter, nt309 | T→G |
| rs12917 | MGMT | MethylGuanine - DNA | 10q26 | Leu84Phe | C→T |
| | | MethylTransferase | | | |
| rs2308321 | MGMT | MethylGuanine - DNA | 10q26 | Ile143Val | A→G |
| | | MethylTransferase | | | |
| rs1799977 | MLH1 | MutL homolog 1 | 3p21.3 | Ile219Val | A→G |
| rs1799750 | MMP1 | Matrix metalloproteinase 1 | 11q22.3 | −1607, promoter | G→GG |
| rs243865 | MMP2 | Matrix metalloproteinase 2 | 16q13-q21 | −1306, promoter | C→T |
| rs1799725 | (SOD2) MnSod | Manganese superoxide dismutase | 6q25.3 | Val16Ala | T→C |
| rs2333227 | MPO | Myeloperoxidase | 17q23.1 | promoter, nt-463 | G→A |
| rs3136229 | MSH6 | Mut S homolog 6 | 2p16 | nt-448, promoter-Sp1 | G→A |
| rs1801133 | MTHFR | 5,10-methylenetetrahydrofolate reductase | 1p36.3 | Ala222Val | C→T |
| | | (NADPH) | | | |
| rs4072037 | MUC1 | Mucin 1 | 1q21 | exon2, splicing | A→G |
| rs1041983 | NAT2 | N-acetylaminotransferase 2 | 8p22 | Tyr94Tyr (nt 282) | C→T |
| rs1801280 | NAT2 | N-acetylaminotransferase 2 | 8p22 | Ile114Th (nt341) | T→C |
| rs1805794 | NBS1 (=NIBRIN) | Nijmegen breakage syndrome 1 (nibrin), | 8q21-q24 | Glu185Gln | G→C |
| | | p95 protein of the MRE11/RAD50 | | | |
| | | complex | | | |
| rs2070744 | NOS | Nitric Oxide Synthase | 7q36 | promoter, nt-786 | T→C |
| rs1052133 | OGG1 | 8-@oxoguanine DNA glycosylase | 3p26.2 | Ser326Cys | C→G |
| rs10895068 | PGR | Progesterone Receptor | 11q22-q23 | promoter, nt+331 | G→A |
| rs1042838 | PGR | Progesterone Receptor (PROGINS) | 11q22-q23 | Val660Leu | G→T |
| rs6917 | PHB | Prohibitin | 17q21 | 3′UTR | C→T |
| rs2233667 | PHB | Prohibitin | 17q21 | intron 5, nt 2582 | C→G |
| rs3856806 | PPARG | Peroxisome proliferator activated receptor γ | 3p25 | nt1431 | C→T |
| rs1801282 | PPARG | Peroxisome proliferator activated receptor γ | 3p25 | Pro12Ala | C→G |
| rs1801270 | P21 | Cyclin dependent kinase inhibitor 1A | 6p21.2 | Ser31Arg | C→A |
| rs2066827 | P27 | Cyclin dependent Kinase inhibitor 1B | 12p13 | Val109Gly | T→G |
| rs1042522 | P53 | Tumor protein p53 | 17p13.1 | Arg72Pro, exon 4 | G→C |
| rs1801173 | P73 | Tumor protein p73 | 1p36.3 | non-coding exon2, | C→T |
| rs13021 | PNN | Pinin | 14q21.1 | Ser671Gly | A→G |
| rs1726801 | POLD1 | DNA Polymerase delta 1 | 19q13.3 | Arg119His | G→A |
| rs1805329 | RAD23B | UV excision repair protein RAD23 | 9q31.2 | Ala249Val | C→T |
| | | homolog B (S. cerevisiae) | | | |
| rs28363284 | RAD51L3 | DNA repair protein RAD51 (S. cerevisiae)- | 17q11 | Glu233Gly | A→G |
| | (=RAD51D) | like 3 | | | |
| rs4796033 | RAD51L3 | DNA repair protein RAD51 (S. cerevisiae)- | 17q11 | Arg165Gln | G→A |
| | (=RAD51D) | like 3 | | | |
| rs3088074 | RAD54 (=ATRX, | Apha thalassemia/mental retardation | 1p32 | Gln929Glu | C→G |
| | RAD54L) | syndrome X-linked | | | |
| rs1799939 | RET | Rearranged during Transfection | 10q11.2 | Gly691Ser | G→A |
| | | protooncogene | | | |
| rs486907 | RNASEL (G/A) | Ribonuclesase L | 1q25 | Arg426Gln | G→A |
| NA | RNASEL (G/T) | Ribonuclease L | 1q25 | Asp541Gln | G→T |
| rs1799941 | SHBG | Sex Hormone Binding Protein | 17p13-p12 | 5′UTR | G→A |
| rs6259 | SHBG | Sex Hormone Binding Protein | 17p13-p12 | Asp356Asn | G→A |
| rs8191979 | SHC1 | SHC Transforming protein 1 | 1q21 | Met300Val | A→G |
| rs 4149396 | SULT1A1 | Sulfotransferase family, cytosolic, 1A, | 16p12.1-p11.2 | Arg213His | G→A |
| | | phenol-preferring, member 1 | | | |
| rs2273535 | STK15 | Serine Threonine protein kinase 15, | 20q | Phe31Ile | T→A |
| | | Aurora Kinase | | | |
| rs3817672 | TFR | Transferrin Receptor | 3q26.2-qter | Ser142Gly | A→G |
| rs1800469 | TGFβ1 | Transforming growth factor, beta 1 | 19q13.1 | promoter, (nt-509) | C→T |
| | | (Camurati-Engelmann disease) | | | |
| NA | TH | Tyrsine hydroxylase | 11p15.5 | nt-4217 | C→T |
| rs1041981 | TNFB | Tumor necrosis factor b | 6p21.3 | Thr26Asn | C→A |
| rs1139793 | TXNR | Thioredoxin Reductase 1 | 12q23-q24.1 | Ile340Thr | C→T |
| rs17868324 | UGT1A7 | UDP glycosyltransferase 1, family, | 2q37 | Arg131Lys | CG→AA |
| | | polypeptide A7 | | | |
| rs11692021 | UGT1A7 | UDP glycosyltransferase 1, family, | 2q37 | Trp208Arg | T→C |
| | | polypeptide A7 | | | |
| rs7975232 | VDR (ApaI) | Vitamin D (1,25-dihydroxyvitamin D3) | 12q12-q14 | ApaI | G→T |
| | | receptor | | | |
| rs1544410 | VDR-BsmI | Vitamin D (1,25-dihydroxyvitamin D3) | 12q12-q14 | intron 7 | G→A |
| | | receptor | | | |
| rs2228570 | VDR-Fok I | Vitamin D (1,25-dihydroxyvitamin D3) | 12q12-q14 | new ATG 5′end | C→T |
| | | receptor | | | |
| rs731236 | VDR-Taq I | Vitamin D (1,25-dihydroxyvitamin D3) | 12q12-q14 | 3′UTR | T→C |
| | | receptor | | | |
| rs3025039 | VEGF | Vascular endothelial growth factor | 6p12 | 3′UTR, nt936 | C→T |
| rs2228000 | XPC | Xeroderma Pigmentosum, | 3p25 | Ala499Val | C→T |
| | | Complementation group C | | | |
| rs2228001 | XPC | Xeroderma Pigmentosum, | 3p25 | Lys939Gln | A→C |
| | | Complementation group C | | | |
| rs17655 | ERCC5 (=XPG) | Xeroderma Pigmentosum | 13q22 | Asp1104His | G→C |
| | | Complementation Group G | | | |
| rs1799782 | XRCC1 | X-ray repair complementing defective | 19q13.2 | Arg194Trp | C→T |
| | | repair in Chinese hamster cells 1 | | | |
| rs25487 | XRCC1 | X-ray repair complementing defective | 19q13.2 | Gln399Arg | A→G |
| | | repair in Chinese hamster cells 1 | | | |
| rs3218536 | XRCC2 | X-ray repair complementing defective | 7q36 | Arg188His | G→A |
| | | repair in Chinese hamster cells 2 | | | |
| rs861539 | XRCC3 | X-ray repair complementing defective | 14q32.3 | Thr241Met | C→T |
| | | repair in Chinese hamster cells 3 | | | |
| rs7830743 | XRCC7 (=PRKDC) | Protein kinase, DNA-activated, catalytic | 8q11 | Ile3433Thr | T→C |
| | | polypeptide | | | |

*rs reference numbers obtained from one of the following sites: world-wide-web at ncbi.nlm.nih.gov/SNP or snp500cancer.nci.nih.gov

TABLE 2
OncoVue ®

| | Parameter Estimate | | |
|---|---|---|---|
| | | Age 30-49 years | |
| | | No 1^{st} | |
| | All Ages | degree | Family history (≧1 first degree |
| | (30-69 yrs) | relative | relative with breast cancer) |
| Intercept | −2.956 | −4.675 | −5.180 |
| SNPs | | | |
| ACACA (IVS17) = T/T | 0.181 | 0.187 | 0.164 |
| ACACA (PIII) = T/T | — | −1.535 | −2.118 |
| CYP11B2 (rs1799998) = T/T | 0.831 | 0.860 | 0.757 |
| CYP1A1 (rs4646903) = C/T or C/C | −0.157 | −0.162 | −0.143 |
| CYP1B1 (rs10012) = C/G or G/G | — | — | 0.525 |
| EPHX (rs1051740) = C/T or T/T | — | — | −0.553 |
| ERCC5 (rs17655) = G/G | −0.836 | −0.864 | −0.610 |
| ESR1 (rs2077647) = C/T or T/T | 1.071 | 1.108 | 0.975 |
| IGF2 (IVS) = G/G | 0.139 | 0.143 | 0.126 |
| MSH6 (rs3136229) = G/G | 0.162 | 0.168 | 0.148 |
| RAD51L3 (rs4796033) = G/G | — | 2.317 | 3.198 |
| SOD2 (rs1799725) = C/T or T/T | — | −1.511 | −2.085 |
| TNFSF6 (rs763110) = C/T or T/T | — | — | 0.371 |
| XPC (rs2228000) = C/T or T/T | — | — | −0.427 |
| Clinical Factors | | | |
| Number of breast biopsies | −0.252 | −0.260 | −0.229 |
| Age (years) at first live birth | −0.226 | −0.234 | −0.206 |
| Parity | — | 2.100 | 2.898 |
| Number of first degree relatives with | −1.219 | −1.260 | −1.110 |
| breast cancer | | | |
| Gail Log Odds Ratio | 2.675 | 2.765 | 2.435 |
| Age Interactions | | | |
| ACACA (5′UTR) = T/T | 0.003 | 0.003 | 0.003 |
| ACACA (PIII) = T/T | — | 0.039 | 0.054 |
| COMT (rs4680) = G/G | — | — | −0.015 |
| CYP11B2 (rs1799998) = T/T | −0.018 | −0.019 | −0.016 |
| CYP19 (rs10046) = C/T or T/T | — | — | 0.012 |
| CYP1B1 (rs1800440) = A/G or G/G | — | −0.004 | −0.006 |
| ERCC5 (rs17655) = G/G | 0.015 | 0.016 | 0.014 |
| ESR1 (rs2077647) = C/T or T/T | −0.020 | −0.020 | −0.018 |
| INS (rs3842752) = C/T or T/T | — | — | 0.011 |
| KLK10 (rs3745535) = G/T or T/T | — | 0.005 | 0.006 |
| RAD51L3 (rs4796033) = G/G | — | −0.057 | −0.078 |
| SOD2 (rs1799725) = C/T or T/T | — | 0.032 | 0.045 |
| VDR (rs7975232) = T/T | 0.003 | 0.003 | 0.003 |
| XRCC2 (rs3218536) = A/G or G/G | 0.016 | 0.017 | 0.015 |
| Parity | — | −0.048 | −0.067 |
| Gail Log Odds Ratio | −0.023 | −0.024 | −0.021 |

*Parameter estimates designated as “—” are not used in group
Bolded parameter estimates are weighted individually and as age interactions = means true if this genotype

TABLE 3
Multifactorial Risk Estimator - Genes, SNPs and Function

| | | SNP ID - | Base | SNP | |
|---|---|---|---|---|---|
| Gene | Gene Name | rs# | Change | Location | Function |
| ACACA | Acetyl coenzyme A carboxylase | N/A | T→C | IVS17 | BRCA1 interaction |
| | alpha | N/A | T→C | 5′UTR | |
| | | N/A | T→G | PIII | |
| | | | | promoter | |
| COMT | Catechol-O-methyltransferase | rs4680 | A→G | V158M | Steroid hormone |
| | | | | | metabolism |
| CYP11B2 | Cytochrome P450, subfamily | rs1799998 | T→C | promoter, | Steroid hormone |
| | XIB, polypeptide 2 | | | nt-344 | metabolism |
| CYP19 | Cytochrome P450, family 19, | rs10046 | T→C | 3′UTR | Steroid hormone |
| | subfamily A, polypeptide 1 | | | | metabolism |
| CYP1A1 | Cytochrome P450, subfamily | rs4646903 | T→C | 3′UTR | Steroid hormone |
| | IA, polypeptide 1 | | | | metabolism |
| CYP1B1 | Cytochrome P450, subfamily | rs1800440 | A→G | N453S | Steroid hormone |
| | IB; polypeptide 1 | rs10012 | C→G | R48G | metabolism |
| EPHX | Epoxide hydrolase | rs1051740 | T→C | Y113H | Xenobiotic metabolism |
| ERCC5 | Excision repair, complementing | rs17655 | G→C | D1104H | DNA repair |
| | defective, in Chinese hamster, 5 | | | | |
| ESR1 | Estrogen receptor 1 | rs2077647 | T→C | S10S | Steroid hormone |
| | | | | | metabolism |
| IGF2 | Insulin-like growth factor II | rs2000993 | G→A | IVS, | Growth |
| | | | | nt3580 | factor/hormone |
| INS | Insulin | rs3842752 | C→T | nt1107 | Growth |
| | | | | | factor/hormone |
| KLK10 | Kallikrein-related peptidase10 | rs3745535 | G→T | A50S | Cell cycle |
| MSH6 | MutS, E. coli homolog of, 6 | rs3136229 | G→A | promoter, | DNA repair |
| | | | | nt-447 | |
| RAD51L3 | RAD51, S. cerevisiae, Homolog | rs4796033 | G→A | R165Q | DNA repair |
| | of, D | | | | |
| SOD2 | Superoxide dismutase 2 | rs1799725 | T→C | V16A | Free radical scavenger |
| TNFSF6 | Tumor necrosis factor ligand | rs763110 | C→T | nt-844 | Apoptosis |
| | superfamily, member 6 | | | | |
| VDR | Vitamin D receptor | rs7975232 | T→G | IVS10 | Hormone receptor |
| XPC | Xeroderma pigmentosum, | rs2228000 | C→T | A499V | DNA repair |
| | complementation group C | | | | |
| XRCC2 | X-ray repair, complementing | rs3218536 | G→A | R188H | DNA repair |
| | defective, in Chinese hamster, 2 | | | | |

TABLE 4
Case Control Ratios

| | | OncoVue ® | | | | | |
|---|---|---|---|---|---|---|---|
| | Risk | (O) | | Gail (G) | | | |
| | Level | n | Ca/Co | n | Ca/Co | O/G | |
| | (%) | (Ca/Co) | Ratio | (Ca/Co) | Ratio | Ratio | Kappa (95% CI) |
| Training (30-44) | >2 | 115/64 | 1.8 | 98/60 | 1.6 | 1.1 | 0.63 (0.56, 0.69) |
| | >3 | 74/28 | 2.6 | 44/26 | 1.7 | 1.5 | 0.50 (0.40, 0.60) |
| Test 1 (30-44) | >2 | 27/18 | 1.5 | 23/18 | 1.3 | 1.2 | 0.66 (0.54, 0.78) |
| | >3 | 13/9 | 1.4 | 10/6 | 1.7 | 0.8 | 0.45 (0.25, 0.65) |
| Test 2 (30-44) | >2 | 8/9 | 0.9 | 2/5 | 0.4 | 2.2 | 0.24 (0.00, 0.49) |
| | >3 | 3/1 | 3.0 | 2/1 | 2.0 | 1.5 | −0.02 (−0.03, −0.004) |
| Training (30-49) | >2 | 421/456 | 0.9 | 423/654 | 0.7 | 1.3 | 0.46 (0.43, 0.50) |
| | >3 | 242/183 | 1.3 | 211/234 | 0.9 | 1.4 | 0.55 (0.51, 0.60) |
| Test 1 (30-49) | >2 | 99/116 | 0.9 | 103/160 | 0.6 | 1.5 | 0.39 (0.31, 0.46) |
| | >3 | 52/42 | 1.2 | 56/56 | 1.0 | 1.2 | 0.62 (0.53, 0.70) |
| Test 2 (30-49) | >2 | 19/46 | 0.4 | 21/56 | 0.4 | 1.0 | 0.42 (0.31, 0.53) |
| | >3 | 10/20 | 0.5 | 9/16 | 0.6 | 0.8 | 0.49 (0.33, 0.64) |
| Training (50-69) | >6 | 496/869 | 0.6 | 414/804 | 0.5 | 1.2 | 0.14 (0.09, 0.17) |
| | >10 | 96/78 | 1.2 | 194/322 | 0.6 | 2.0 | 0.31 (0.27, 0.36) |
| Test 1 (50-69) | >6 | 117/209 | 0.6 | 97/168 | 0.6 | 1.0 | 0.20 (0.12, 0.30) |
| | >10 | 21/12 | 1.8 | 43/63 | 0.7 | 2.6 | 0.32 (0.21, 0.42) |
| Test 2 (50-69) | >6 | 37/61 | 0.6 | 27/52 | 0.5 | 1.2 | 0.24 (0.10, 0.40) |
| | >10 | 3/3 | 1.0 | 11/19 | 0.6 | 1.7 | 0.12 (−0.03, 0.28) |
| Training (30-69) | >12 | 270/242 | 1.1 | 454/760 | 0.6 | 1.8 | 0.34 (0.31, 0.37) |
| | >20 | 132/52 | 2.5 | 145/168 | 0.9 | 2.8 | 0.45 (0.39, 0.51) |
| Test 1 (30-69) | >12 | 70/50 | 1.4 | 118/160 | 0.7 | 2.0 | 0.10 (0.03, 0.17) |
| | >20 | 30/11 | 2.7 | 37/32 | 1.2 | 2.2 | 0.45 (0.33, 0.57) |
| Test 2 (30-69) | >12 | 20/15 | 1.3 | 25/46 | 0.5 | 2.6 | 0.21 (0.09, 0.33) |
| | >20 | 1/3 | 0.3 | 3/7 | 0.4 | 0.8 | 0.28 (−0.03, 0.59) |

TABLE 5
Fold Stratification

| | | OncoVue ® | | Gail | | |
|---|---|---|---|---|---|---|
| | | Range | Difference | Range | Difference | Ratio |
| Training (30-44) | | | | | | |
| Cases | High to Low | 55.04-0.20 | 54.83 | 10.14-0.49 | 9.65 | 5.68 |
| | 95^{th}-5^{th} percentile | 7.43-0.46 | 6.98 | 4.31-0.58 | 3.73 | 1.87 |
| Controls | High to Low | 12.41-0.14 | 12.27 | 9.07-0.49 | 8.57 | 1.43 |
| | 95^{th}-5^{th} percentile | 2.14-0.37 | 1.78 | 2.22-0.54 | 1.68 | 1.06 |
| Test 1 (30-44) | | | | | | |
| Cases | High to Low | 15.92-0.37 | 15.55 | 9.07-0.49 | 8.58 | 1.81 |
| | 95^{th}-5^{th} percentile | 6.99-0.54 | 6.45 | 4.55-0.54 | 4.00 | 1.61 |
| Controls | High to Low | 18.79-0.04 | 18.75 | 8.83-0.49 | 8.34 | 2.25 |
| | 95^{th}-5^{th} percentile | 2.35-0.36 | 1.99 | 2.50-0.60 | 1.90 | 1.04 |
| Test 2 (30-44) | | | | | | |
| Cases | High to Low | 3.56-0.35 | 3.21 | 4.67-0.49 | 4.17 | 0.77 |
| | 95^{th}-5^{th} percentile | 3.01-0.57 | 2.45 | 1.61-0.56 | 1.05 | 2.33 |
| Controls | High to Low | 10.39-0.23 | 10.16 | 3.33-0.49 | 2.83 | 3.59 |
| | 95^{th}-5^{th} percentile | 2.19-0.42 | 1.77 | 1.56-0.49 | 1.07 | 1.66 |
| Training (30-49) | | | | | | |
| Cases | High to Low | 71.17-0.34 | 70.83 | 19.06-0.98 | 18.09 | 3.92 |
| | 95^{th}-5^{th} percentile | 11.49-0.91 | 10.58 | 8.04-1.21 | 6.82 | 1.55 |
| Controls | High to Low | 20.71-0.20 | 20.52 | 20.29-0.98 | 19.32 | 1.06 |
| | 95^{th}-5^{th} percentile | 4.58-0.72 | 3.86 | 4.72-1.07 | 3.65 | 1.06 |
| Test 1 (30-49) | | | | | | |
| Cases | High to Low | 28.32-0.57 | 27.75 | 17.14-0.98 | 16.16 | 1.72 |
| | 95^{th}-5^{th} percentile | 13.02-0.93 | 12.09 | 8.75-1.07 | 7.68 | 1.57 |
| Controls | High to Low | 39.49-0.09 | 39.41 | 16.71-0.98 | 15.73 | 2.51 |
| | 95^{th}-5^{th} percentile | 4.33-0.74 | 3.60 | 4.98-1.12 | 3.87 | 0.93 |
| Test 2 (30-49) | | | | | | |
| Cases | High to Low | 8.33-0.48 | 7.85 | 9.02-0.98 | 8.04 | 0.98 |
| | 95^{th}-5^{th} percentile | 5.56-0.86 | 4.70 | 4.81-1.07 | 3.74 | 1.26 |
| Controls | High to Low | 14.25-0.35 | 13.90 | 6.47-0.98 | 5.49 | 2.53 |
| | 95^{th}-5^{th} percentile | 3.36-0.79 | 2.57 | 3.04-0.99 | 2.05 | 1.25 |
| Training (50-69) | | | | | | |
| Cases | High to Low | 25.53-1.63 | 23.89 | 63.71-3.41 | 60.29 | 0.40 |
| | 95^{th}-5^{th} percentile | 15.81-4.17 | 11.64 | 19.27-3.74 | 15.53 | 0.75 |
| Controls | High to Low | 25.24-1.64 | 23.60 | 49.13-3.41 | 45.72 | 0.52 |
| | 95^{th}-5^{th} percentile | 9.69-3.71 | 5.98 | 15.56-3.74 | 11.82 | 0.51 |
| Test 1 (50-69) | | | | | | |
| Cases | High to Low | 19.94-2.81 | 17.13 | 56.06-3.41 | 52.65 | 0.33 |
| | 95^{th}-5^{th} percentile | 13.98-4.03 | 9.94 | 16.70-3.74 | 12.95 | 0.77 |
| Controls | High to Low | 19.34-2.28 | 17.06 | 34.08-3.41 | 30.67 | 0.56 |
| | 95^{th}-5^{th} percentile | 9.23-3.94 | 5.29 | 14.59-3.74 | 10.85 | 0.49 |
| Test 2 (50-69) | | | | | | |
| Cases | High to Low | 11.66-4.16 | 7.50 | 16.56-3.41 | 13.15 | 0.57 |
| | 95^{th}-5^{th} percentile | 10.01-4.69 | 5.32 | 12.61-3.74 | 8.86 | 0.60 |
| Controls | High to Low | 11.66-2.81 | 8.85 | 23.61-3.41 | 20.20 | 0.44 |
| | 95^{th}-5^{th} percentile | 8.86-3.72 | 5.14 | 13.87-3.41 | 10.45 | 0.49 |
| Training (30-69) | | | | | | |
| Cases | High to Low | 77.32-2.35 | 74.97 | 71.79-4.33 | 67.45 | 1.11 |
| | 95^{th}-5^{th} percentile | 25.52-5.34 | 20.18 | 24.45-4.75 | 19.70 | 1.02 |
| Controls | High to Low | 39.21-1.66 | 37.55 | 59.56-4.33 | 55.23 | 0.68 |
| | 95^{th}-5^{th} percentile | 13.12-4.91 | 8.22 | 20.01-4.75 | 15.26 | 0.54 |
| Test 1 (30-69) | | | | | | |
| Cases | High to Low | 45.24-2.81 | 42.42 | 65.68-4.33 | 61.34 | 0.69 |
| | 95^{th}-5^{th} percentile | 26.22-5.42 | 20.80 | 24.77-4.75 | 20.02 | 1.04 |
| Controls | High to Low | 49.16-2.35 | 46.80 | 44.36-4.33 | 40.02 | 1.17 |
| | 95^{th}-5^{th} percentile | 12.51-4.88 | 7.63 | 17.12-4.75 | 12.37 | 0.62 |
| Test 2 (30-69) | | | | | | |
| Cases | High to Low | 22.74-4.31 | 18.43 | 24.46-4.33 | 20.13 | 0.92 |
| | 95^{th}-5^{th} percentile | 13.83-5.82 | 8.01 | 16.33-4.75 | 11.58 | 0.69 |
| Controls | High to Low | 21.32-3.41 | 17.91 | 28.11-4.33 | 23.78 | 0.75 |
| | 95^{th}-5^{th} percentile | 11.85-4.86 | 6.99 | 13.64-4.33 | 9.31 | 0.75 |

TABLE 6
Discriminatory Accuracy

| | % Predictive Above Random | |
|---|---|---|
| | Chance (95% CI), p-value | |
| Sample Set | OncoVue ® | Gail |
| Ages 30-44 | | |
| Training (533 Ca/1048 Co) | 17 (13, 19), <0.0001 | 8 (5, 11), <0.0001 |
| Test 1 (127 Ca/271 Co) | 14 (8, 20), <0.0001 | 7 (0.8, 13), 0.02 |
| Test 2 (48 Ca/154 Co) | 17 (8, 26), <0.0004 | 13 (4, 22), 0.007 |
| Ages 30-49 | | |
| Training (834 Ca/1661 Co) | 15 (13, 18), <0.0001 | 8 (5, 10), <0.0001 |
| Test 1 (202 Ca/410 Co) | 13 (8, 18), <0.0001 | 8 (3, 13), 0.002 |
| Test 2 (85 Ca/224 Co) | 18 (11, 25), <0.0001 | 14 (7, 21), 0.0001 |
| Ages 50-69 | | |
| Training (837 Ca/1690 Co) | 8 (5, 10), <0.0001 | 2 (−1, 4), 0.21 |
| Test 1 (198 Ca/383 Co) | 4 (−1, 9), 0.15 | 5 (0, 10), 0.058 |
| Test 2 (57 Ca/140 Co) | 14 (5, 22), 0.0026 | 9 (0.2, 17), 0.054 |
| Ages 30-69 | | |
| Training (1671 Ca/3351 Co) | 9 (7, 11), <0.0001 | 4 (2, 6), <0.0001 |
| Test1 (400 Ca/793 Co) | 8 (4, 11), <0.0001 | 6 (2, 10), 0.00014 |
| Test 2 (142 Ca/364 Co) | 14 (9, 20), <0.0001 | 11 (5, 16), 0.0002 |

TABLE 7
Difference in Discriminatory Accuracy

| | % Average |
|---|---|
| | Improvement (95% CI), p-value |
| Sample Set | OncoVue ® vs. Gail |
| Ages 30-44 | |
| Training (533 Ca/1048 Co) | 52 (35, 70), <0.0001 |
| Test 1 (127 Ca/271 Co) | 100 (14, 185), 0.018 |
| Test 2 (48 Ca/154 Co) | 24 (−87, 95), 0.50 |
| Ages 30-49 | |
| Training (834 Ca/1661 Co) | 50 (36, 65), <0.0001 |
| Test 1 (202 Ca/410 Co) | 40 (3, 78), 0.033 |
| Test 2 (85 Ca/224 Co) | 20 (−31, 64), 0.38 |
| Ages 50-69 | |
| Training (837 Ca/1690 Co) | 80 (43, 118), <0.0001 |
| Test 1 (198 Ca/383 Co) | 3 (−15, 20), 0.70 |
| Test 2 (57 Ca/140 Co) | 36 (−60, 110), 0.34 |
| Ages 30-69 | |
| Training (1671 Ca/3351 Co) | 59 (40, 79), <0.0001 |
| Test 1 (400 Ca/793 Co) | 39 (−31, 103), 0.23 |
| Test 2 (142 Ca/364 Co) | 26 (−19, 67), 0.23 |

TABLE 8
Fold Improvement in the PLRs at the 12% Risk Threshold

| | PLR (95% CI)* | | Fold | |
|---|---|---|---|---|
| | Multifactorial | | Improvement | |
| Sample Set | Risk Estimator | Gail Model | (95% CI) | p-value |
| Training | 2.1 (1.8, 2.5) | 1.2 (1.1, 1.3) | 1.8 (1.4, 2.2) | <0.0001 |
| Test 1 | 2.4 (1.7, 3.3) | 1.5 (1.2, 1.8) | 1.7 (1.1, 2.5) | 0.024 |
| Test 2 | 3.2 (1.8, 5.6) | 1.4 (1.0, 2.2) | 2.2 (1.1, 5.3) | 0.034 |
| Blinded | 2.2 (1.1, 4.3) | 0.90 (0.6, 1.3 | 2.4 (1.1, 5.6) | 0.036 |
| Validation | | | | |

*PLR = Positive likelihood ratio, CI = Confidence Interval

TABLE 9
Breast Cancer Cases at Fixed Gail Model Control Levels (12% risk)

| | | | | | | Percent |
|---|---|---|---|---|---|---|
| | | | | | No. of | more |
| | | | | | Additional | Cases |
| | Gail Model | | OncoVue ® | | Detected | over |
| Sample Set | Cases | Controls | Cases | Controls | Cases | Gail |
| Training | 454 | 760 | 577 | 760 | 123 | 27% |
| Test 1 | 118 | 161 | 135 | 161 | 17 | 14% |
| Test 2 | 32 | 56 | 42 | 56 | 10 | 31% |
| Blinded | 37 | 43 | 56 | 43 | 19 | 51% |
| Validation | | | | | | |

### Example 3

### Conclusion

In summary, the inventors have examined genetic polymorphisms in a number of genes and have determined their association with breast cancer risk. The unexpected results of these experiments were that, considered individually, the examined genes and their polymorphisms were only modestly associated with breast cancer risk. However, when examined in combination of two, three or more, complex genotypes with wide variation in breast cancer risk were identified. This information has great utility in facilitating the most effective and most appropriate application of cancer screening and chemoprevention protocols, with resulting improvements in patient outcomes.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### IX. REFERENCES

The following references, to the extent that they provide exemplary procedural or other details supplementary to those set forth herein, are specifically incorporated herein by reference.
U.S. Pat. No. 4,683,195
U.S. Pat. No. 4,683,202
U.S. Pat. No. 4,800,159
U.S. Pat. No. 4,883,750
U.S. Pat. No. 5,578,832
U.S. Pat. No. 5,837,832
U.S. Pat. No. 5,837,860
U.S. Pat. No. 5,861,242
U.S. Pat. No. 6,159,693
Aston et al., Hum Genet. 116(3):208-21, 2005 (Epub 2004 Dec. 21).
Bailey et al., Cancer Res., 58(22):5038-5041, 1998.
Benichou and Gail, Biometrics, 46:991-1003, 1990.
Benichou and Gail, Biometrics, 51:182-194, 1995.
Bergman-Jungestrom et al., Int JCancer 84:350-53, 1998.
Bruzzi et al., Am J. Epidemiol., 122:904-914, 1995.
Clarke et al., Breast Cancer Res., 4(6):R13, 2002.
Connell et al., Mol. Cell. Endocrinol., 217(1-2):243-247, 2004.
Costantino et al., J. Natl. Cancer Inst., 91:1541-1548, 1999.
Cox and Hinkley, Theoretical Statistics, Wiley, NY, 1974.
De Vivo et al., Breast Cancer Res 6:R636-39, 2004.
Ebell Evidence-based diagnosis: a handbook of clinical prediction rules, Springer, New York, N.Y., 2001.
Efron and Gong, The American Statistician, 1983.
European Patent Appln. 320,308
European Patent Appln. 329,822
Fodor et al., Biochemistry, 30(33):8102-8108, 1991.
Frohman, In: PCR Protocols: A Guide To Methods And Applications, Academic Press, N.Y., 1990.
Gail et al. J. Natl. Cancer Inst., 81:1879-1886, 1989 GB Appln. 2,202,328
Guyatt and Rennie, eds. Users' guide to the medical literature: evidence-based clinical practice, American Medical Association Press, Chicago, Ill., 2002.
Hacia et al., Nature Genet., 14:441-449, 1996.
Hartl and Clark, Principles of Population Genetics, Sinauer Associates, Inc., Sunderland, Mass., 1997.
Holmstrom et al., Anal. Biochem. 209:278-283, 1993.
Hosmer and Lemeshow, Applied Logic Regression, 2d Ed., Wiley, NY, 2002.
Innis et al., Proc. Natl. Acad. Sci. USA, 85(24):9436-9440, 1988.
Jupe et al., Lancet 357:1588-89, 2001.
Kwoh et al., Proc. Natl. Acad. Sci. USA, 86:1173, 1989.
Lander and Schork, Science, 30:265:2037-2048, 1994.
Magnard et al., Oncogene, 21(44):6729-6739, 2002.
McTiernan et al., Cancer Epidemiol Biomarkers Prev., 10:333-338, 2001.
Nelson et al., Breast Cancer Res 7:R357-64, 2005.
Newton et al., Nucl. Acids Res., 21:1155-1162, 1993.
Ohara et al., Proc. Natl. Acad. Sci. USA, 86:5673-5677, 1989.
PCT Appln. PCT/US87/00880
PCT Appln. PCT/US89/01025
PCT Appln. WO 2003/025141
PCT Appln. WO 2005/024067
PCT Appln. WO 88/10315
PCT Appln. WO 89/06700
PCT Appln. WO 90/07641
Pease et al., Proc. Natl. Acad. Sci. USA, 91:5022-5026, 1994.
Ralph et al., Cancer, 109:1940-1948, 2007.
Rasmussen et al., Anal. Biochem., 198:138-142, 1991.
Reddy and Chow, Am. J. Health Syst. Pharm., 57:1315-2132, 2000.
Rockhill et al., J Natl Cancer Inst., 93:358-366, 2001.
Running et al., Bio Techniques 8:276-277, 1990.
Sambrook et al., In: Molecular Cloning: A Laboratory Manual, 2d Ed., Cold Spring Harbor Laboratory Press, Cold Spring Harbor, N.Y., 2001.
Surveillance, Epidemiology, and End Results (SEER) Program (world-wide-web at seer.cancer.gov) DevCan database: “SEER 13 Incidence and Mortality, 2000-2002, Follow-back year=1992, with Kaposi Sarcoma and Mesothelioma”. National Cancer Institute, DCCPS, Surveillance Research Program, Cancer Statistics Branch, released April 2005, based on the November 2004 submission. Underlying mortality data provided by NCHS (world-wide-web at cdc.gov/nchs).
Shoemaker et al., Nature Genetics, 14:450-456, 1996.
Sinilnikova et al., Carcinogenesis, 25:2417-2424, 2004.
Spurdle et al., Cancer Epidemiol. Biomarkers Prev., 11(5):439-443, 2002.
Thompson et al., Cancer Res 58:2107-10, 1998.
Walker et al., Nucleic Acids Res., 20(7):1691-1696, 1992.
Wedren et al., Carcinogenesis 24:681-87, 2003.
Wrensch et al., Breast Cancer Res., 5(4):R88-102, 2003.
Wu and Wallace, Genomics, 4:560-569, 1989.
Ye et al., Hum. Mutat., 17(4):305-16, 2001.
Zhu et al., Breast Cancer Res 7:R745-R752, 2005.

What is claimed is:

1. A method for assessing a female subject's risk for developing breast cancer comprising determining, in a sample from said subject, the allelic profile of more than one SNP selected from the group consisting of ACACA (IVS17) T→C, ACACA (5′UTR) T→C, ACACA (PIII) TAG, COMT (rs4680) A→G, CYP19 (rs10046) T→C, CYP1A1 (rs4646903) T→C, CYP1B1 (rs1800440) A→G, EPHX (rs1051740) T→C, TNFSF6 (rs763110) C→T, IGF2 (rs2000993) G+A, INS (rs3842752) C→T, KLK10 (rs3745535) G→T, MSH6 (rs3136229) G→A, RAD51L3 (rs4796033) G→A, XPC (rs2228000) C→T, and XRCC2 (rs3218536) G→A.

2. The method of claim 1, further comprising determining the allelic profile of each SNP in claim 1.

3. The method of claim 1, further comprising determining the allelic profile of at least one additional SNP selected from the group consisting of CYP11B2 (rs1799998) T→C, CYP1B1 (rs10012) C→G, ESR1 (rs2077647) T→C, SOD2 (aka MnSOD, rs1799725) T→C, VDR ApaI (rs7975232) T→G, and ERCC5 (rs17655) G→C.

4. The method of claim 3, further comprising determining the allelic profile of each SNP in claim 3.

5. The method of claim 1, further comprising assessing one or more aspects of the subject's personal history.

6. The method of claim 1, wherein said one or more aspects are selected from the group consisting of age, ethnicity, reproductive history, menstruation history, use of oral contraceptives, body mass index, alcohol consumption history, smoking history, exercise history, diet, family history of breast cancer or other cancer including the age of the relative at the time of their cancer diagnosis, and a personal history of breast cancer, breast biopsy or DCIS, LCIS, or atypical hyperplasia.

7. The method of claim 6, wherein one or more aspects comprises age.

8. The method of claim 1, wherein determining said allelic profile is achieved by amplification of nucleic acid from said sample.

9. The method of claim 8, wherein amplification comprises PCR.

10. The method of claim 8, wherein primers for amplification are located on a chip.

11. The method of claim 8, wherein primers for amplification are specific for alleles of said genes.

12. The method of claim 8, further comprising cleaving amplified nucleic acid.

13. The method of claim 8, wherein said sample is derived from oral tissue or blood.

14. The method of claim 1, further comprising making a decision on the timing and/or frequency of cancer diagnostic or screening testing for said subject.

15. The method of claim 1, further comprising making a decision to place said subject on advanced cancer diagnostic or screening testing.

16. The method of claim 1, further comprising making a decision on the timing and/or frequency of prophylactic cancer treatment for said subject.

17. A nucleic acid microarray comprising nucleic acid sequences corresponding to genes at least one of the alleles for each of ACACA (IVS17) T→C, ACACA (5′UTR) T→C, ACACA (PIII) T→G, COMT (rs4680) A→G, CYP19 (rs10046) T→C, CYP1A1 (rs4646903) T→C, CYP1B1 (rs1800440) A→G, EPHX (rs1051740) T→C, TNFSF6 (rs763110) C→T, IGF2 (rs2000993) G→A, INS (rs3842752) C→T, KLK10 (rs3745535) G→T, MSH6 (rs3136229) G→A, RAD51L3 (rs4796033) G→A, XPC (rs2228000) C→T, and XRCC2 (rs3218536) G→A.

18. The nucleic acid microarray of claim 17, wherein said nucleic acid sequences comprise sequences for both alleles for each of said genes.

19. A method for determining the need for routine diagnostic testing of a female subject for breast cancer comprising determining, in a sample from said subject, the allelic profile of more than one SNP selected from the group consisting of ACACA (IVS17) T→C, ACACA (5′UTR) T→C, ACACA (PIII) T→G, COMT (rs4680) A→G, CYP19 (rs10046) T→C, CYP1A1 (rs4646903) T→C, CYP1B1 (rs1800440) A→G, EPHX (rs1051740) T→C, TNFSF6 (rs763110) C→T, IGF2 (rs2000993) G→A, INS (rs3842752) C→T, KLK10 (rs3745535) G→T, MSH6 (rs3136229) G→A, RAD51L3 (rs4796033) G→A, XPC (rs2228000) C→T, and XRCC2 (rs3218536) G→A.

20. A method for determining the need of a female subject for prophylactic anti-breast cancer therapy comprising determining, in a sample from said subject, the allelic profile of more than one SNP selected from the group consisting of ACACA (IVS17) T→C, ACACA (5′UTR) T→C, ACACA (PIII) T→G, COMT (rs4680) A→G, CYP19 (rs10046) T→C, CYP1A1 (rs4646903) T→C, CYP1B1 (rs1800440) A→G, EPHX (rs1051740) T→C, TNFSF6 (rs763110) C→T, IGF2 (rs2000993) G→A, INS (rs3842752) C→T, KLK10 (rs3745535) G→T, MSH6 (rs3136229) G→A, RAD51L3 (rs4796033) G→A, XPC (rs2228000) C→T, and XRCC2 (rs3218536) G→A.
